(19) United States
(12) Patent Application Publication
(10) Pub. No.: US 2004/0162517 A1
(43) Pub. Date: Aug. 19, 2004

(54) Needleless hydpodermic injection device with non-electric ignition means

(76) Inventors: Otto Furst, Viernheim (DE); Hans-Peter Haar, Wiesloch (DE); George Bevan Kirby Meacham, Shaker Heights, OH (US); Hans List, Hesseneck (DE)

Correspondence Address: HOFFMANN-LA ROCHE INC., PATENT LAW DEPARTMENT, 340 KINGSLAND STREET, NUTLEY, NJ, 07110

(21) Appl. No.: 10/720,908

(22) Filed: Nov. 24, 2003

Related U.S. Application Data
(60) Provisional application No. 60/430,783, filed on Dec. 4, 2002.

(51) Int. Cl. A61M 5/30

(52) U.S. Cl. 604/69

(57) ABSTRACT

A device for performing a needleless hypodermic injection of a liquid medication contained in the device. The device includes pyrotechnical means for generating within the device a pressure necessary for injecting the medication. The device comprises non-electric ignition means for igniting a propellant contained in said device. The ignition means comprise in particular an impact sensitive primer material and a firing pin for striking said primer material, said primer material being so positioned with respect to the propellant that when the firing pin strikes the primer material the hot products of combustion of said primer material ignite the propellant.

### PRIORITY TO PROVISIONAL APPLICATION(S) UNDER 35 U.S.C. § 119(e)

This application claims priority under 35 U.S.C. §119(e) of provisional application Serial No. 60/430,783, filed on Dec. 4, 2002.

### FIELD OF THE INVENTION

The invention concerns a device for performing a needleless hypodermic injection of a liquid medication contained in the device, said device including pyrotechnical means for generating within the device a pressure necessary for injecting the medication, said device comprising ignition means for igniting a propellant contained in said device.

Within the scope of the instant invention a propellant is a pyrotechnic fuel which mainly contributes to the delivery of thermal energy and gas production of a pyrotechnic system.

Within the scope of the instant invention an ignition material is a pyrotechnic material used in a pyrotechnic initiator.

### BACKGROUND OF THE INVENTION

U.S. Pat. No. 6,258,063 describes a needleless hypodermic injection device of the above-mentioned kind comprising electric ignition means for igniting a propellant contained in said device and thereby generating the gas pressure necessary for performing the injection. Electric ignition requires the use of batteries as source of energy. Use of batteries is disadvantageous, because disposal of batteries in normal trash is not allowed because of environmental concerns. Thus electric ignition is not a suitable solution for a needleless injection device that should be disposable in normal trash after a single or at most a few injections performed therewith. Moreover it is wasteful to discard the entire injection device with its battery after a single use, because the battery could still be used for performing more than a single injection.

### SUMMARY OF THE INVENTION

In view of the above mentioned drawbacks of electric ignition the aim of the invention is to provide needleless injection devices of the above-mentioned kind which include non-electric ignition means and which therefore operate battery-free.

According to a first aspect of the invention the above aim is achieved with a needleless hypodermic injection device wherein the ignition means comprise an impact initiated primer material and a firing pin for striking said primer material, said primer material being so positioned with respect to said propellant that when the firing pin strikes the primer material the hot products of combustion of said primer material ignite said propellant.

According to a second aspect of the invention the above aim is achieved with a needleless hypodermic injection device wherein the ignition means comprise

(a) a first body having a cavity, said cavity having an outlet through which the interior of the cavity is fluidically connected with a zone within the device where said propellant is located, said first body being deformable by bending it,

(b) a second body arranged within said cavity so that a portion of the outer surface of the second body is spaced from a portion of the inner surface of said cavity, said second body being to some extent elastically deformable by bending it, but adapted to be broken when that bending exceeds a predetermined threshold value, and

(c) said portion of the outer surface of the second body and said portion of the inner surface of said cavity facing each other and one or both of those surface portions being coated with an impact sensitive pyrotechnic material, whereby an impact of said surface portions on each other caused by breaking the second body by bending of the first body and thereby bending of the cavity that contains the second body beyond said threshold value ignites said impact sensitive pyrotechnic material and the products of this ignition ignite the propellant.

According to a third aspect of the invention the above aim is achieved with a needleless hypodermic injection device wherein the ignition means comprise

(a) a first body having a cavity, said cavity having an outlet through which the interior of the cavity is fluidically connected with a zone within the device where said propellant is located, the tubular portion of said first body being deformable by a localized impact from one side,

(b) a solid anvil arranged within said tube so that a portion of the outer surface of the anvil is spaced from a portion of the inner surface of said tube, and

(c) means for effecting a mechanical impact on a spot of the outer surface of said first body, said portion of the outer surface of the anvil and said portion of the inner surface of said tube facing each other and one or both of those surface portions being coated with an impact sensitive pyrotechnic material, whereby an impact of said surface portions on each other effected by said means for effecting a mechanical impact ignites said pyrotechnic material and the products of this ignition ignite the propellant.

According to a fourth aspect of the invention the above aim is achieved with a needleless hypodermic injection device wherein the ignition means comprise

(a) a pair of hypergolic members formed by a first and a second hypergolic member which are contained within the device, but separated from each other,

said hypergolic members being able to chemically react with each other when brought into contact with each other and which thereby generate heat for igniting said propellant, and

(b) means for bringing said first and a second hypergolic member into contact with each other.

In a first preferred embodiment of this kind of device the ignition means comprise

(a) a first tube having a closed end and an open end through which the interior of the tube is fluidically connected with a zone within the device where said propellant is located, said tube being deformable by bending it, and

(b) a second tube closed at both ends and arranged within said first tube so that at least a portion of the outer surface of the second tube is spaced from a portion of the inner surface of said first tube, said second tube being breakable by bending the first tube when that bending exceeds a predetermined threshold value,

said second tube containing a first member of an hypergolic pair, and

(c) a second member of an hypergolic pair contained in a space between the inner surface of the first tube and the outer surface of the second tube, whereby when said second tube is broken by bending of the first tube beyond said threshold value the first member and the second member of the hypergolic pair contact each other and their chemical reaction causes an ignition and the products of this ignition ignite the propellant.

In an alternative to the embodiment just described above the ignition means comprise

(a) an elongated container having a closed end and an open end through which the interior of the container is fluidically connected with a zone within the device where said propellant is located, said container being deformable by bending it,

(b) a first tube closed at both ends and arranged within said container, said first tube containing a first member of an hypergolic pair, and

(c) a second tube closed at both ends and arranged within said container, said second tube containing a second member of an hypergolic pair,

said first and second tubes being breakable by bending the container when that bending exceeds a predetermined threshold value, whereby when the first and second tubes are broken by bending of the container beyond said threshold value the first member and the second member of the hypergolic pair contact each other and their chemical reaction causes an ignition and the products of this ignition ignite the propellant.

According to a fifth aspect of the invention the above aim is achieved with a needleless hypodermic injection device wherein the ignition means comprise

(a) a rotatable body,

(b) a stationary body,

(c) means for pressing at least a portion of said rotatable body against said stationary body, a portion of the outer surface of said rotatable body being thereby in contact with said stationary body and

at least a part of said portion of the outer surface of said rotatable body or at least a part of the outer surface of said stationary body being coated with a combustible mixture that bursts into flame when activated by friction,

(d) locking means that when locked prevent rotation of said rotatable body,

(e) means for rotating said rotatable body when said locking means are unlocked, said rotation causing a friction of said combustible mixture against an outer surface of said stationary body or an outer surface of said stationary body and thereby ignition of said mixture, the products of this ignition igniting the propellant.

According to a sixth aspect of the invention the above aim is achieved with a needleless hypodermic injection device wherein the ignition means comprise

(a) a rotatable body,

(b) a stationary body,

(c) means for pressing at least a portion of said rotatable body against said stationary body, a portion of the outer surface of said rotatable body being thereby in contact with said stationary body and

at least a part of said portion of the outer surface of said rotatable body being coated with a first member of an hypergolic pair, and at least a part of the outer surface of said stationary body being coated with a second member of an hypergolic pair,

(d) locking means that when locked prevent rotation of said rotatable body,

(e) means for rotating said rotatable body when said locking means are unlocked, said rotation bringing said first and second hypergolic member into contact with each other, thereby causing their chemical reaction and a resulting ignition, the products of this ignition igniting the propellant.

According to a seventh aspect of the invention the above aim is achieved with a needleless hypodermic injection device wherein the ignition means comprise a piezoelectric spark generator.

The main advantage obtained with a needlefree injection device according to the invention is that it is battery-free and can therefore be disposed in household waste. Moreover some of the non-electric ignition means of a device according to the invention make it possible to achieve a significant reduction of the manufacturing cost of the device.

Each of the above mentioned embodiments of the invention is suitable for being combined e.g. with one of the following examples of structures of a needle free injection device:

### EXAMPLE 1

A first structure of a needle free injection device comprises

(a) a housing,

(b) a first chamber within said housing, said first chamber containing a medication unit configured and dimensioned to store a volume of liquid medication to be injected, said medication unit having a first region and a second region that are in liquid communication with each other, said first region being deformable and said second region having an ejection outlet, and

(c) a second chamber within said housing, said second chamber containing a propellant,

said first chamber comprising two zones, a first zone containing said medication unit and a second zone which is in communication with said second chamber, so that upon ignition of the propellant in the second chamber gas generated thereby expands into said second zone of said first chamber, exerts pressure on and deforms said deformable first region of said medication unit and thereby causes ejection of said liquid medication through said ejection outlet.

### EXAMPLE 2

A second structure of a needle free injection device comprises

(a) a nozzle body, and

(b) a rigid housing,

said housing having a first open end adapted to receive and be connected with the nozzle body and a second closed end,

the interior of said housing defining a chamber which extends between said open end and said closed end of the housing, said chamber being adapted to receive

a first deformable diaphragm which together with a cavity of said nozzle body forms a medication chamber suitable for receiving a predetermined amount of a medication, and

a second deformable diaphragm a portion of which extends around a portion of said first deformable diaphragm,

said second deformable diaphragm and said housing forming together a chamber for receiving a propellant and means for igniting said propellant, and

said nozzle body having at its outer end an orifice which is the outlet of a channel for ejecting said medication out of said chamber when a gas pressure generated by ignition of said propellant is applied to said second deformable diaphragm and thereby to said first deformable diaphragm.

### EXAMPLE 3

A third structure of a needle free injection device comprises

(a) a rigid medication container having a medication zone for receiving said liquid medication,

(b) a nozzle in fluidic communication with said medication zone, said nozzle having an outlet orifice,

(c) a propellant zone where said propellant is located within said device,

(d) a channel that fluidically connects said propellant zone with said medication zone, and

(e) piston means slidably arranged within said channel, so that upon ignition of said propellant gas pressure generated by combustion of the propellant causes displacement of said piston means which then exert pressure on said liquid medication and eject it through the outlet orifice of said nozzle.

### BRIEF DESCRIPTION OF THE DRAWINGS

The subject of the invention will now be described in terms of its preferred embodiments with reference to the accompanying drawings. These embodiments are set forth to aid the understanding of the invention, but are not to be construed as limiting.

FIG. 1 shows a cross-sectional view of a first embodiment of an injection device 11 according to the invention.

FIG. 2 shows an exploded cross-sectional view of components of device 11 shown by FIG. 1.

FIG. 3 shows a view of device 11 similar to FIG. 1, but in reduced scale.

FIGS. 4 and 5 show exploded cross-sectional perspective views of device 11 from different points of view.

FIGS. 6 and 7 show assembled cross-sectional perspective views of device 11 corresponding to FIGS. 4 and 5 respectively.

FIGS. 8 to 12 show the structure of an application device 49 used in combination with injection device 11 of FIGS. 1-7.

FIGS. 13 to 16 illustrate the operation of application device 49 of FIGS. 8 to 12.

FIG. 17 shows a partial cross-sectional view of an injection device having a second embodiment of ignition means.

FIG. 18 shows a cross-sectional view of igniter 61 in FIG. 17.

FIG. 19 shows a cross-sectional view of a slight bending of igniter 61.

FIG. 20 shows a cross-sectional view of a stronger bending of igniter 61 that breaks rod 65 contained in tube 64.

FIG. 21 shows a perspective view of igniter 61 inserted in ignition plate 62.

FIG. 22 shows an exploded perspective view of the assembled components shown by FIG. 21.

FIGS. 23 and 24 are similar to FIGS. 21 and 22, but show views taken from a different point of view.

FIG. 25 shows a partial cross-sectional view of an injection device having a third embodiment of ignition means.

FIG. 26 shows a cross-sectional view of igniter 81 in FIG. 25.

FIG. 27 shows a perspective view of igniter 81 inserted in ignition plate 62.

FIG. 28 shows an exploded perspective view of the assembled components shown by FIG. 27.

FIGS. 29 and 30 are similar to FIGS. 27 and 28, but show views taken from a different point of view.

FIGS. 31 to 33 show perspective views of a injection device and a spring mechanism connected thereto which is arranged for impacting on a spot of the outer surface of igniter 81.

FIGS. 34 to 37 show various embodiments of needle free injection devices which can be combined with the ignition means described with reference to FIGS. 25 to 33.

FIG. 38 shows a partial cross-sectional view of an injection device having a fourth embodiment of ignition means.

FIG. 39 shows a cross-sectional view of igniter 131 in FIG. 38.

FIG. 40 shows a cross-sectional view of a bending of tube 134 that breaks ampoule 135 contained in tube 134.

FIG. 41 shows a perspective view of igniter 131 inserted in ignition plate 62.

FIG. 42 shows an exploded perspective view of the assembled components shown by FIG. 41.

FIGS. 43 and 44 are similar to FIGS. 41 and 42, but show views taken from a different point of view.

FIG. 45 shows a partial cross-sectional view of an injection device having ignition means similar to those of the above mentioned fourth embodiment.

FIG. 46 shows a cross-sectional view of igniter 151 in FIG. 45.

FIG. 47 shows a cross-sectional view of a bending of container 154 that breaks ampoules 155 and 157 contained in container 154.

FIG. 48 shows a perspective view of igniter 151 inserted in ignition plate 62.

FIG. 49 is similar to FIG. 48, but show a view taken from a different point of view.

FIG. 50 shows an exploded perspective view of the assembled components shown by FIGS. 48 and 49 respectively.

FIG. 51 shows a partial cross-sectional view of an injection device having a fifth embodiment of ignition means.

FIG. 52 shows a perspective view of the above described igniter arrangement assembled with ignition plate 62.

FIG. 53 shows an exploded perspective view of the components shown by FIG. 52.

FIGS. 54 and 55 are similar to FIGS. 52 and 53, but show views taken from a different point view.

FIG. 56 shows a partial cross-sectional view of an injection device having a sixth embodiment ignition means.

FIG. 57 shows a schematic cross-sectional view of an embodiment of a needleless injector module containing a one-piece propellant pellet.

FIG. 58 shows a one-piece propellant pellet.

FIGS. 59-61 show several arrays comprising several one-piece propellant pellets.

### REFERENCE NUMERALS IN DRAWINGS

| | |
|---|---|
| 1 | |
| 2 | primer/primer material |
| 3 | |
| 4 | |
| 5 | firing pin |
| 6 | hollow rim |
| 7 | shell case |
| 8 | |
| 9 | moisture seal |
| 10 | |
| 11 | needleless injection device/needleless |
| | injection cartridge |
| 12 | medication container |
| 13 | medication unit |
| 14 | flexible wall of medication container 12 |
| 15 | nozzle |
| 16 | fluid channel |
| 17 | orifice/jet outlet |
| 18 | deformable/elastic barrier |
| 19 | break-off protective cap |
| 20 | first housing part |
| 21 | second housing part |
| 22 | |
| 23 | propellant container |
| 24 | propellant |
| 24a | propellant inside shell case 7 |
| 24b | propellant outside shell case 7 |
| 25 | |
| 26 | |
| 27 | |
| 28 | intermediate support member |
| 29 | |
| 30 | screw connection of housing parts |
| 31 | first chamber |
| 32 | second chamber |
| 33 | first zone of first chamber |
| 34 | second zone of first chamber |
| 35 | burst membrane/zone of reduced thickness |
| | of wall of propellant container |
| 36 | |
| 37 | |
| 38 | |
| 39 | |
| 40 | |
| 41 | plunger housing |
| 42 | plunger/impact plunger |
| 43 | spring/mainspring |
| 44 | latch fingers |
| 45 | assembly latches |
| 46 | cam interface/release ramp |
| 47 | push shell |
| 48 | |
| 49 | application or actuation device/impact |
| | ignition actuation device |
| 50 | |
| 51 | sense spring |
| 52 | safety latch |
| 53 | endcap/plastic cap |
| 54 | |
| 55 | |
| 56 | |
| 57 | |
| 58 | centering lug |
| 59 | centering lug |
| 60 | |
| 61 | glass snap rod igniter |
| 61 | ignition plate |
| 63 | propellant chamber |
| 64 | tube |
| 65 | rod |
| 66 | impact sensitive ignition material coating |
| 67 | closed end of tube 64 |
| 68 | open end portion of tube 64 |
| 69 | retaining crimp |
| 70 | air gap |
| 71 | outlet of tube 64 |
| 72 | frangible moisture seal |
| 73 | secondary ignition material |
| 74 | |
| 75 | |
| 76 | |
| 77 | |
| 78 | centering lug |
| 79 | retaining crimp |
| 80 | air gap |
| 81 | igniter |
| 82 | spot in ignition impact zone |
| 83 | |
| 84 | tube |
| 85 | metallic anvil pin |
| 86 | impact sensitive ignition material coating |
| 87 | end part of anvil pin 85 |
| 88 | open end portion of tube 84 |
| 89 | closed end of tube 84 |
| 90 | |
| 91 | outlet of tube 84 |
| 92 | frangible moisture seal |
| 93 | secondary ignition material |
| 94 | |
| 95 | |
| 96 | |
| 97 | |
| 98 | |
| 99 | |
| 100 | |
| 101 | torsion spring coil |
| 102 | L-arm of spring 101 |
| 103 | release latch |
| 104 | pivot notch |
| 105 | trigger guard |
| 106 | trigger guard |
| 107 | spring limit stop |
| 108 | support |
| 109 | |
| 110 | |
| 111 | one-piece propellant |
| 112 | ignition layer |
| 113 | nozzle body |
| 114 | rigid housing |
| 115 | deformable diaphragm |
| 116 | deformable diaphragm |
| 117 | orifice |
| 118 | channel of nozzle body 113 |
| 119 | medication chamber |
| 120 | peel off foil seal tab |
| 121 | rigid medication container |
| 122 | medication zone |
| 123 | nozzle |
| 124 | orifice |
| 125 | propellant zone |
| 126 | propellant |
| 127 | channel |
| 128 | piston |
| 129 | housing part |
| 130 | |
| 131 | igniter |
| 132 | |
| 133 | |
| 134 | first body/metallic tube |
| 135 | second tube/breakable ampoule |
| 136 | first hypergolic member |
| 137 | second hypergolic member |
| 138 | open end portion of tube 134 |
| 139 | retaining crimp |
| 140 | |
| 141 | outlet of tube 134 |
| 142 | frangible moisture seal |
| 143 | secondary ignition material |
| 144 | |
| 145 | |
| 146 | |
| 147 | |
| 148 | |
| 149 | |
| 150 | |
| 151 | igniter |
| 152 | |
| 153 | |
| 154 | container/metallic tube |
| 155 | first tube/breakable ampoule |
| 156 | first hypergolic fluid |
| 157 | second tube/breakable ampoule |
| 158 | second hypergolic fluid |
| 159 | |
| 160 | |
| 161 | outlet of container 154 |
| 162 | frangible moisture seal |
| 163 | secondary ignition material |
| 164 | |
| 165 | |
| 166 | |
| 167 | |
| 168 | |
| 169 | |
| 170 | |
| 171 | rotatable body/rotatable match |
| 172 | stationary body/striker ring |
| 173 | compression-torsion-spring/pressing |
| | means/driving means |
| 174 | locking pin/actuation pull pin |
| 175 | driving means/anchor post |
| 176 | end of spring 173 in a snap lock |
| 177 | stop flange |
| 178 | |
| 179 | |
| 180 | |
| 181 | piezoelectric spark generator |
| 182 | electrode |
| 183 | electrode |
| 184 | end part of electrode |
| 185 | end part of electrode |
| 186 | actuation button of piezoelectric generator |
| 187 | |
| 188 | |
| 189 | |
| 190 | |
| 191 | nozzle body |
| 192 | housing |
| 193 | deformable diaphragm |
| 194 | cavity |
| 195 | medication chamber |
| 196 | deformable diaphragm |
| 197 | propellant/propellant pellet |
| 198 | ignition layer |
| 199 | orifice |
| 200 | |
| 201 | nozzle channel/injection channel |
| 202 | removable foil seal |
| 203 | venting holes |
| 204 | ignition pins |
| 205 | screw connection |
| 206 | ignition plate |
| 207 | hole in propellant pellet 197 |
| 208 | |
| 209 | |
| 210 | |
| 211 | stack of one-piece propellant pellets |
| 212 | one-piece propellant pellet |
| 213 | one-piece propellant pellet |
| 214 | one-piece propellant pellet |
| 215 | hole extending through propellant |
| | pellets 212-214 |
| 216 | array of concentric cylindrical one-piece |
| | propellant pellets |
| 217 | one-piece propellant pellet |
| 218 | one-piece propellant pellet |
| 219 | one-piece propellant pellet |
| 220 | hole extending through array 216 |
| 221 | array one-piece propellant pellets |
| 222 | one-piece propellant pellet |
| 223 | one-piece propellant pellet |
| 224 | one-piece propellant pellet |
| 225 | one-piece propellant pellet |
| 226 | one-piece propellant pellet |
| 227 | one-piece propellant pellet |
| 228 | radius |
| 229 | radius |
| 230 | radius |
| 231 | hole extending through array 221 |

### DETAILED DESCRIPTION OF PREFERRED EMBODIMENTS

Various embodiments of a device according to the invention for performing a needleless hypodermic injection of a liquid medication contained in the device are described hereinafter with reference to the accompanying figures. Each of these embodiments comprises pyrotechnical means for generating within the device a pressure necessary for injecting the medication. For this purpose, the embodiments described hereinafter comprise non-electric ignition means for igniting a propellant contained in the device.

Embodiment 1

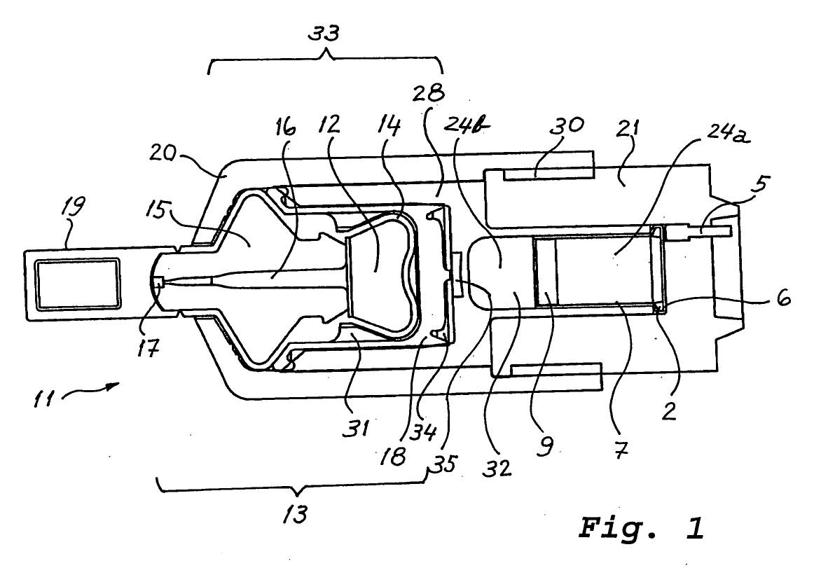

FIG. 1 shows a first embodiment of a device 11 according to the invention. Device 11 is a cartridge characterized in that the ignition means contained therein comprise an impact sensitive primer material 2 and a firing pin 5 for striking primer material 2. Primer material 2 is so positioned with respect to a propellant 24a lodged within a shell case 7 that when a firing pin 5 strikes primer material 2 the hot products of combustion of primer material 2 ignite propellant 24a contained in shell case 7 and optionally an additional propellant 24b contained within the device.

Shell case 7 and primer material 2 are parts of a gas generator which is part of device 11. Shell case 7 seals the rear of this gas generator and no other gas seal is needed for that purpose.

Shell case 7 is a thin brass cup that has a hollow rim 6 around the base. Hollow rim 6 of shell case 7 is filled with impact-sensitive primer material 2. Filling of primer material 2 in rim 6 is done by putting a wet primer material in the cup and then spinning it so that centrifugal force moves the mix into the rim. When a firing pin mechanism is actuated firing pin 5 strikes and crushes rim 6 at one point and thereby ignites primer material 2.

Primer material 2 is preferably a “green igniter” of the type applied to powerloads for pyrotechnically actuated devices such as nail guns that are used in the workplace to avoid pollution problems due to toxic heavy metals contained in “non-green” igniters. “Green” igniters or powerloads are manufactured for pyrotechnically powered tools.

One of the important advantages of the above described device 11 is that it can be manufactured at low cost, in particular because the manufacture of a powerload comprising shell case 7 and primer material 2 filled in rim 6 of shell case 7 uses technology and production capacity established to serve the small-arms ammunition and powerload markets. The powerload is e.g. a caliber 22 rimfire cartridge. The powerload comprising shell case 7, primer material 2 and optionally an additional propellant within shell case 7 may be purchased as a sealed, assembled module that includes all the required propellant. This strongly simplifies the process for manufacturing injection device 11.

Ignition of primer material 2 generates a certain amount of hot gas on its own, and lights additional propellant contained in the shell case.

Additional propellant optionally contained within device 11 is e.g. a fine grain nitrocellulose based composition or another nitrocellulose based composition, or another propellant composition having similar properties or a mixture of propellant compositions.

Shell case 7—or in general the container of primer material 2—is preferably dosed by a moisture seal 9 that opens at low pressure and that excludes contact of the primer material with water vapor prior to use of the device. After ignition of the primer and propellant and opening of the moisture seal 9 at low pressure, the propellant continues to burn in the volume comprised between the cartridge head and an optional burst diaphragm 35 that separates the gas generator from the cartridge head. This volume may contain additional propellant of the same or a different type.

When the pressure reaches a predetermined level, the burst diaphragm opens and the pressure generated by combustion of the primer and propellant causes ejection of the medication out of the device for performing an injection.

Firing pin 5 is held captive in a hole in a housing part of device 11. The mechanism for actuating firing pin 5 comprises a plunger (not shown) that drives pin 5 into the shell case rim 6. A safety rim shields pin 5 from accidental impact during the manufacturing process.

In a variant of this embodiment, the impact initiated primer material 2 is part of a centerfire primer arrangement consisting of a deformable cup, an anvil, and primer material 2 in which firing pin 5 indents the cup and crushes the primer material 2 against the anvil when actuated.

In a further preferred embodiment, ignition of primer material 2 alone produces sufficient gas volume and pressure that no additional propellant is needed to perform an injection with device 11.

In a further preferred embodiment, ignition of primer material 2 produces a first, fast high pressure pulse that starts the injection, and at the same time ignites a slower burning propellant that provides a lower pressure over a longer duration for completing the injection.

Shell case 7 has an open end. In a preferred embodiment, the open end of the shell case 7 is sealed with a frangible closure, e.g. moisture seal 9 that excludes penetration of water vapor into the shell case prior to use of the device.

The propellant must not necessarily be entirely contained within shell case 7. In a preferred embodiment, at least a portion of propellant 24a is contained within shell case 7 and another portion 24b of propellant 24 is located outside shell case 7. The portion of propellant 24a located within shell case 7 has preferably properties which are different from the properties of the portion of propellant 24b located outside shell case 7.

In a preferred embodiment, primer material 2 is sealed with a frangible closure 9 that excludes contact of primer material 2 with water vapor prior to use of device 11.

Injection device 11 described above can have different structures.

FIG. 1 shows a cross-sectional view of a first example of a structure of device 11 designed as a single use device, that is a device which is used only once and discarded after use.

Injection device 11 shown by FIG. 1 comprises a housing consisting of a first housing part 20, e.g. an aluminum shell, and a second housing part 21, made e.g. of a polycarbonate. Housing parts 20 and 21 have threads which match with each other and are thus be connected with each other by a screw connection 30.

In a preferred embodiment the housing of device 11 is so configured and dimensioned that as a whole is adapted to withstand an internal pressure which is higher than the normal injection pressure without yielding.

In a preferred embodiment, both parts 20 and 21 of the housing of device 11 are made of suitable plastic materials, e.g. from commercially available polyesters or polycarbonates taking in particular into account the mechanical properties the housing should have.

The interior of the housing of device 11 comprises a first chamber 31 and a second chamber 32, which are defined for instance by respective cavities of a support member 28.

Support member 28 is made preferably of a rigid, plastic material which does rather break than yield when subject to mechanical stress. Support member 28 is made e.g. of thermoplastic polyester or a polycarbonate having the above mentioned properties.

A medication unit 13 comprising nozzle 15 and medication container 12 formed by deformable wall 14 is arranged within first chamber 31. A volume of liquid to be injected is stored in medication unit 13. In preferred embodiments, the amount of this volume is in a range going from about 50 to 1000 microliters. Specific examples of this amount are e.g. 200 or 500 microliters.

Medication unit 13 is a sealed medication module which comprises a nozzle body 15 and a flexible container wall 14 that hermetically encloses a portion of the nozzle and forms a reservoir 12 for a liquid medication stored in sealed medication unit 13. Wall 14 is deformable and collapsible.

Medication unit 13 thus comprises a first region and a second region that are in liquid communication with each other. The first region is deformable and comprises the reservoir enclosed by flexible wall 14. The second region of medication unit 13 comprises nozzle 15 which has a fluid channel 16 that ends in an orifice 17 which serves as a liquid jet outlet through which liquid to be injected is ejected when an injection is performed with injector module 11. Medication unit 13 is made of one or more suitable construction materials, e.g. polyethylene and polypropylene, which are suitable for storing medications including sensitive protein drugs.

A part of container wall 14 forms a break-off protective cap 19 that covers a jet orifice 17 of nozzle body 15. Cap 19 is removed by the user just prior to use of injector module 11.

First chamber 31 comprises two zones, a first zone 33 which contains medication unit 13 and a second zone 34 which is located between medication unit and second chamber 32.First chamber 31 is in communication with second chamber 32 so that upon ignition of propellant 24 located within second chamber 32, gas thereby generated expands into second zone 34 of first chamber 31, exerts pressure on and deforms deformable wall 14 of the first region of medication unit 13 and thereby causes ejection of the liquid medication through channel 16 and orifice 17.

In a preferred embodiment a deformable/elastic barrier 18 divides the first zone 33 from the second zone 34. The elastic barrier is made e.g. of silicon rubber, and can be reinforced e.g. with woven aramid fibers.

Support member 28 has a wall that separates the first chamber 31 from the second chamber 32. This has a zone of reduced thickness 35 that has the function of a burst membrane which bursts and gives way when the pressure within the second chamber 32 exceeds a predetermined value.

In a preferred embodiment the free volume comprised between medication unit 13 and the wall of support member 28 with burst membrane 35 is much smaller than the volume available for containing propellant within the device.

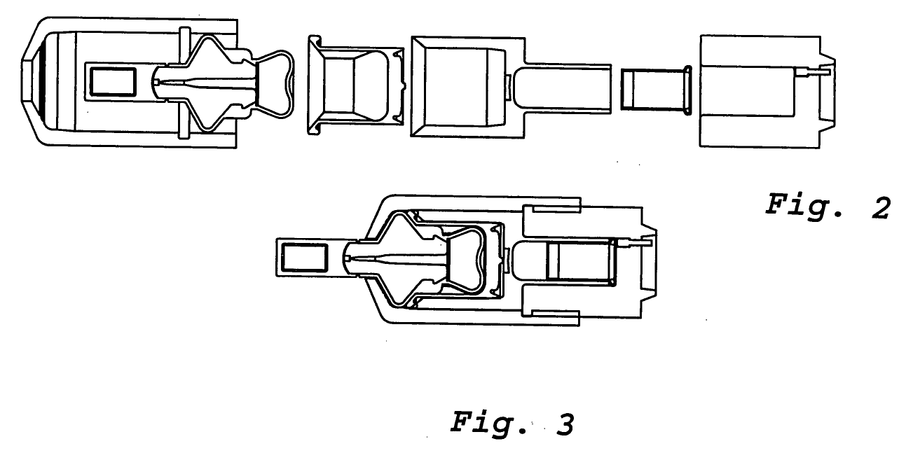

FIG. 2 shows an exploded cross-sectional view of components of device 11 shown by FIG. 1.

FIG. 3 shows a view of device 11 similar to FIG. 1, but in reduced scale.

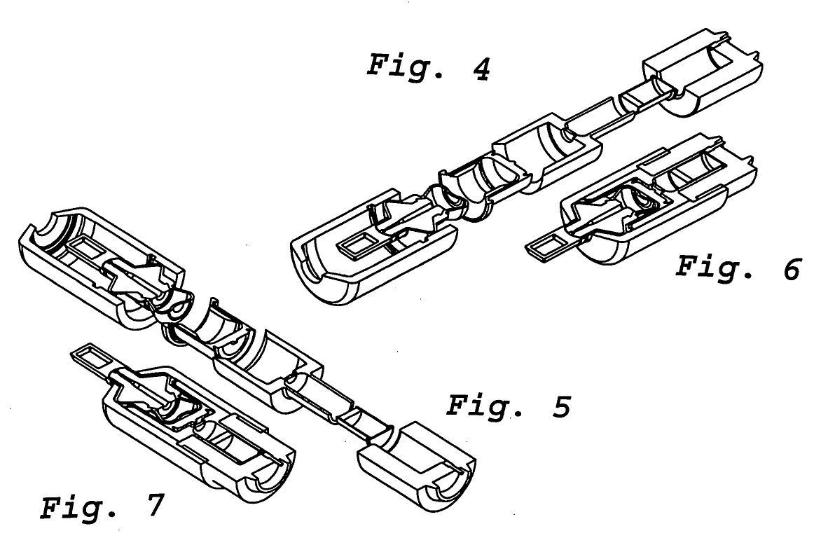

FIGS. 4 and 5 show exploded cross-sectional perspective views of device 11 from different points of view.

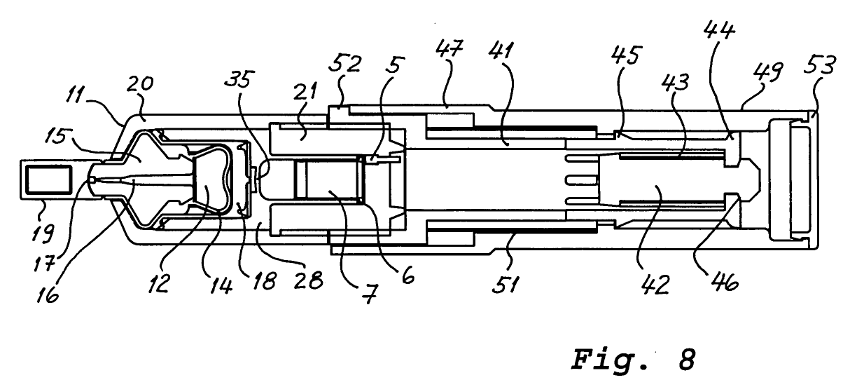

FIGS. 6 and 7 show assembled cross-sectional perspective views of device 11 corresponding to FIGS. 4 and 5 respectively.

Other examples of structures of injection devices are described hereinafter in the description of other embodiments comprising different ignition means. The ignition means described above with reference to FIGS. 1 to 7 are also suitable for those other examples of structures of injection devices.

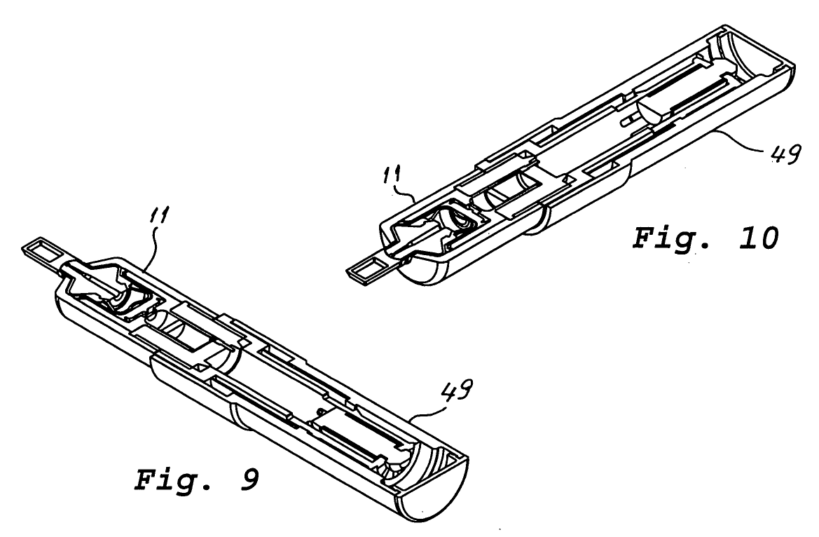

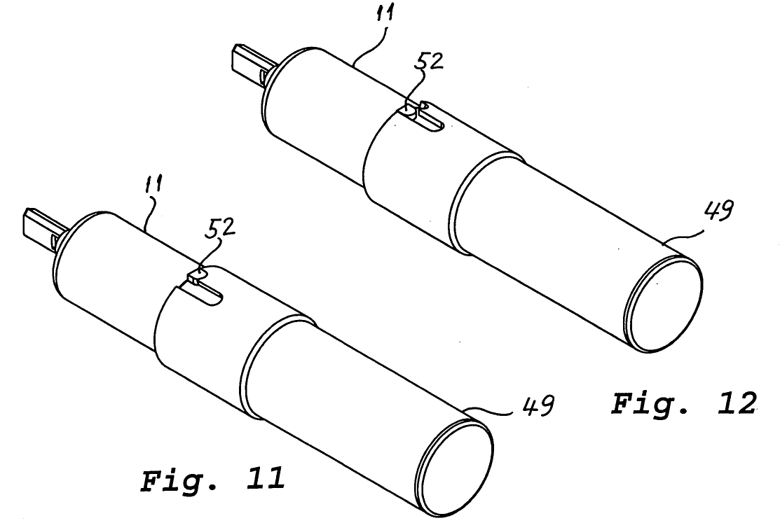

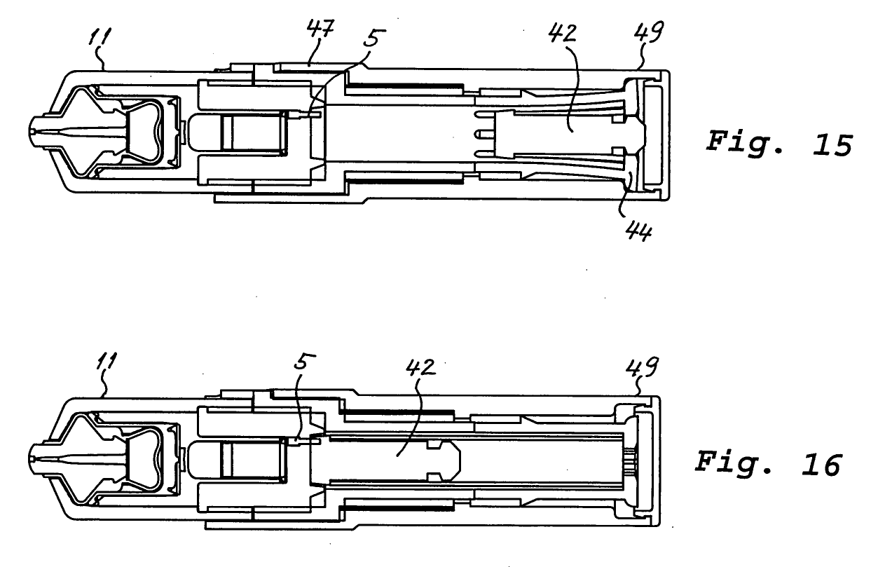

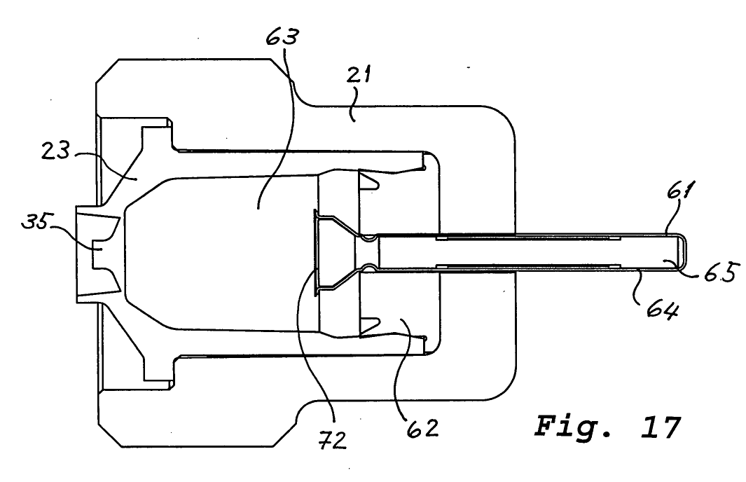

An injection device 11 of the type described above is used in combination with an application device 49 which contains a firing pin mechanism having a plunger 42 for striking the firing pin which drives pin 5 into the shell case rim 6. An example of such an application device 49 is described hereinafter with reference to FIGS. 8 to 16.

Application device 49 comprises a plunger 42 which is driven by a spring 43 for striking firing pin 5 with an energy of about 170 millijoules and a trigger mechanism operated by pressing the end of the injection device 11 where the orifice 17 for liquid ejection is located against the injection site. Application device 49 is manufactured as a separate module that is screwable to the rear of device 11.

The structure of application device 49 is shown in FIGS. 8 to 12.

As shown by FIG. 8 application device 49 has a molded plastic plunger housing 41 that contains an impact plunger 42 and a mainspring 43 and guides impact plunger 42 from its initial cocked position to impact on firing pin 5. Plunger housing 41 is slotted to form latch fingers 44 that engage and hold impact plunger 42 in the cocked position. Housing 41 also includes a set of assembly latches 45 that retain a push shell 47 that slides over plunger housing 41.

Impact plunger 41 is e.g. a steel plunger that is slidably arranged inside plunger housing 41. Impact plunger 41 is urged toward firing pin 5 by compressed mainspring 43 and retained in a cocked position by latch fingers 44. Mainspring 43 is a catalog part with 0.55 mm wire diameter, 7.62 mm outer diameter, and a free length of 50.8 mm.

A cam interface 46 between latch fingers 44 and impact plunger 42 causes force provided by mainspring 43 to push out latch fingers 44 radially. In the cocked position latch fingers 44 are restrained by push shell 47 and retain plunger 42.

Push shell 47 which is e.g. a molded plastic shell forms the outer housing of application device 49. Push shell 47 snaps in place over plunger housing 41 and is preloaded towards the rear by sense spring 51. Sense spring 51 determines the force the user must apply to trigger injection device and thereby effect an injection.

A projecting safety latch 52 on plunger housing 41 engages a matching notch in push shell 47 and thereby prevents shell 47 from sliding and releasing impact plunger 42 accidentally. Safety latch 52 is released just prior to injection by twisting push shell 47 clockwise.

A molded plastic endcap 53 is snapped into push shell 47 to close the end of application device 49. Endcap 53 is not necessary for the function of application module 49 and is provided only for aesthetic purpose.

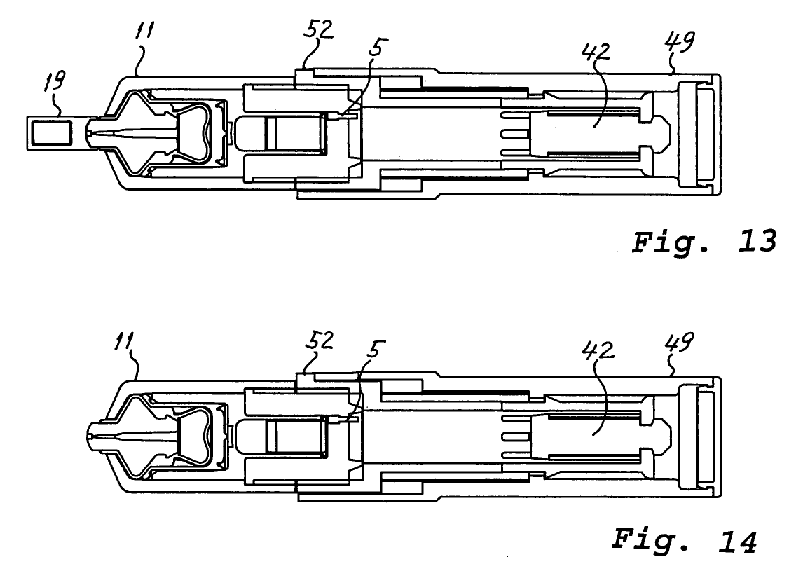

The operation of application device 49 as a mechanism for producing an impact on firing pin 5 of injection device 11 is described hereinafter with reference to FIGS. 13 to 16.

FIG. 13 shows the assembled injection device 11 and application device 49 as shipped. Application device 49 is shipped with safety latch 52 engaged and removable protective cap 19 covering the nozzle.

Just before use, protective cap 19 is removed and push shell 47 is rotated to release safety latch 52. At this point, push shell 47 is free to slide over plunger housing 41 and release latch fingers 44.

To make the injection, the user grasps push shell 47 and presses the nozzle 15 of injection device 11 against the injection site. Push shell 47 slides forward, and latch fingers 44 approach an annular release ramp 46 in the rear of push shell 47. The force the user has to apply is primarily determined by sense spring 51 up to this point.

When latch fingers 44 reach release ramp 46, they are cammed out by the motion of impact plunger 42 and mainspring 43 as plunger 42 is released. The interaction of the radial motion of latch fingers 44 with release ramp 46 moves push shell 47 further forward. The user perceives this as an overcenter snap-through indicating injector actuation.

After impact plunger 42 is released as described above, it is accelerated by the mainspring until it reaches and strikes firing pin 5 which thereby strikes and ignites the powerload (primer material 2) to make the injection.

Application device can be a single use device or a reusable impact ignition actuation device designed to be recocked for each use.

As can be appreciated from the foregoing description, the application device 49 is a snap-together assembly of plastic moldings, wire springs, and a steel plunger. These components are low in cost, and the structure of application device 49 is suitable for high speed automated assembly thereof. After application device 49 is fully assembled and inspected, it is connected to injection device 11 by a screw connection. Safety and user friendly features are included in the design.

As shipped, push shell 47 of application device 49 is locked in the rear position thereof. This positively holds latch fingers 44 in engagement with impact plunger 42 and makes accidental actuation thereof very unlikely even if injection device 11 is dropped.

The slidable push shell 47 and sense spring 51 require a deliberate motion for actuation, and the overcenter action at the instant plunger 42 is released provides a tactile signal to the user that the injection is taking place.

Embodiment 2

FIGS. 17 to 24 show a second embodiment of ignition means suitable for use as part of an injection device having a structure similar to the structure of the above described injection device 11 in FIG. 1.

Other examples of structures of injection devices are described hereinafter in the description of other embodiments comprising different ignition means. The ignition means described below with reference to FIGS. 17 to 24 are also suitable for those other examples of structures of injection devices.

FIG. 17 shows a partial view of an injection device having a similar structure as injection device 11 shown in FIG. 1, but wherein a glass snap rod igniter 61 replaces the firing pin 5 and the impact sensitive primer material 2 shown in FIG. 1 as ignition means.

Igniter 61 is inserted and held in a suitable opening of an ignition plate 62 that is a gas-tight closure of the rear part of a propellant chamber 63.

Igniter 61 comprises a first body, e.g. a metal tube 64, which is deformable, can be bent, and the interior of which is a cavity which is fluidically connected with propellant chamber 63.

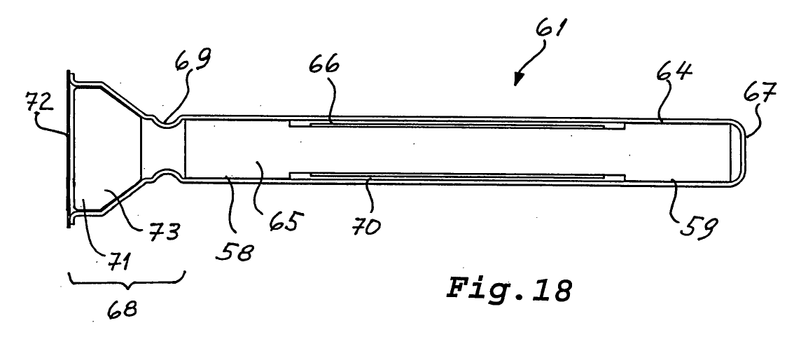

As can be appreciated from FIG. 18 which shows the structure of igniter 61 more in detail, a second body, e.g. a glass snap rod 65 is arranged within metal tube 64 so that a portion of the outer surface of rod 65 is spaced from and is in face of a portion of the inner surface of tube 64. There is an air gap 70 between the latter surface portions. Centering lugs 58 and 59 at both ends of rod 65 ensure the spacing between those surface portions. Lugs 58 and 59 have slots that provide axial passages for gas flow.

Rod 65 is to some extent elastically deformable and can be bent, but is broken when bending thereof exceeds a predetermined threshold value.

One or both of the above-mentioned surface portions that face each other is/are coated with an impact sensitive pyrotechnic material 66. In the example shown by FIG. 18, the outer surface of the middle zone of rod 65 is coated with a layer of impact sensitive pyrotechnic material 66. In a preferred embodiment, an annular groove around the periphery of rod 65 defines a fracture point.

In a preferred embodiment one of those surface portions which face each other is coated with an impact sensitive pyrotechnic material and the other is coated with a secondary ignition material.

In another preferred embodiment one or both of those surface portions which face each other is coated with a layer of an impact sensitive pyrotechnic material and a layer of a secondary ignition material.

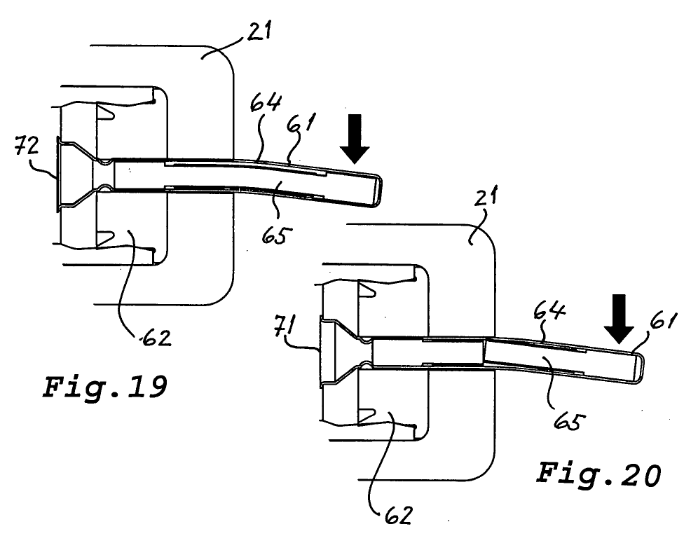

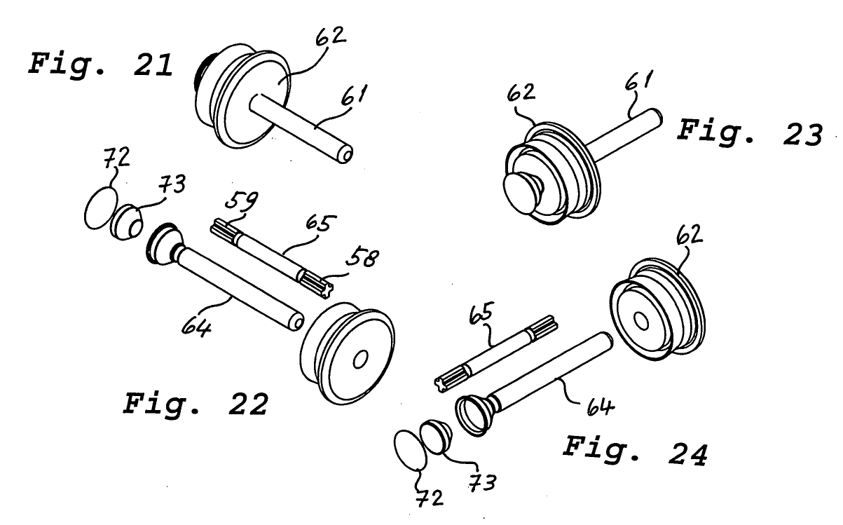

When tube 64 is slightly bent as shown by FIG. 19, both tube 64 and rod 65 contained therein are elastically deformed and store mechanical energy, but when tube 64 and thereby rod 65 are bent beyond a predetermined threshold value, rod 65 is broken as shown by FIG. 20 at its fracture point and the above mentioned surface portions of tube 64 and rod 65 impact on each other and this ignites the impact sensitive pyrotechnic material coated on one or both of those surface portions. The products of this ignition, hot gas and sparks, ignite a propellant contained in propellant chamber 63. Bending of tube 64 is effected by means of an actuation button or the like.

As shown by FIG. 18, tube 64 has a dosed end and an open, funnel shaped end portion 68 that end in an outlet 71 open towards the propellant chamber 63. Tube 64 has a retaining crimp 69 that keeps rod 65 in a predetermined axial position within tube 64 and retain rod pieces when rod 65 is broken.

In a preferred embodiment outlet 71 is sealed with a frangible moisture seal 72 that excludes penetration of water vapor into the interior of tube 64 prior to use of igniter 61.

In a further preferred embodiment, a secondary ignition material 73, e.g. a primer mix, is provided and located outside of tube 64 near the outlet 71 of tube 64, so that products of combustion of the impact sensitive pyrotechnic material 66 ignite the secondary ignition material and thereby increase the amount of hot material for igniting the propellant in propellant chamber 63.

In another preferred embodiment, a secondary ignition material 73, e.g. a primer mix, is provided and located inside of tube 64 and near the outlet 71 of tube 64, so that products of combustion of the impact sensitive pyrotechnic material ignite the secondary ignition material 73 and thereby increase the amount of hot material for igniting the propellant.

FIG. 21 shows a perspective view of igniter 61 inserted in ignition plate 62. FIG. 22 shows an exploded perspective view of the assembled components shown by FIG. 21.

FIGS. 23 and 24 are similar to FIGS. 21 and 22, but show views taken from a different point of view.

Embodiment 3

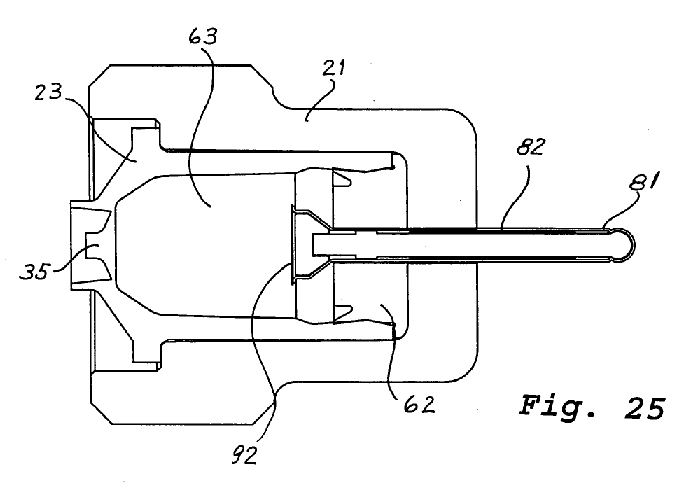

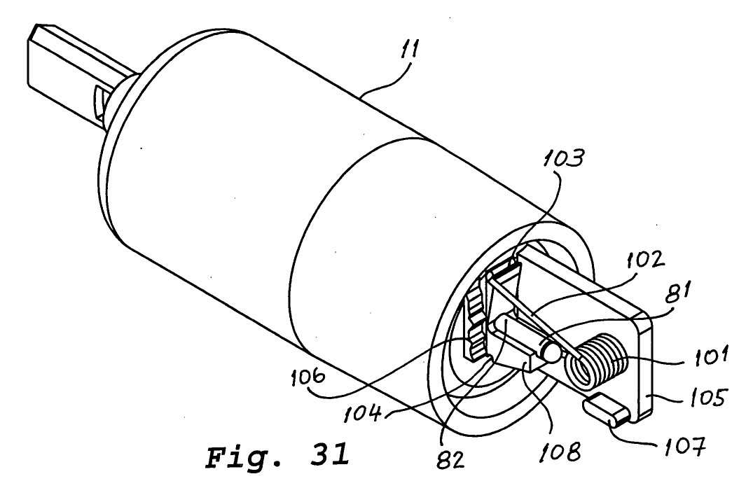

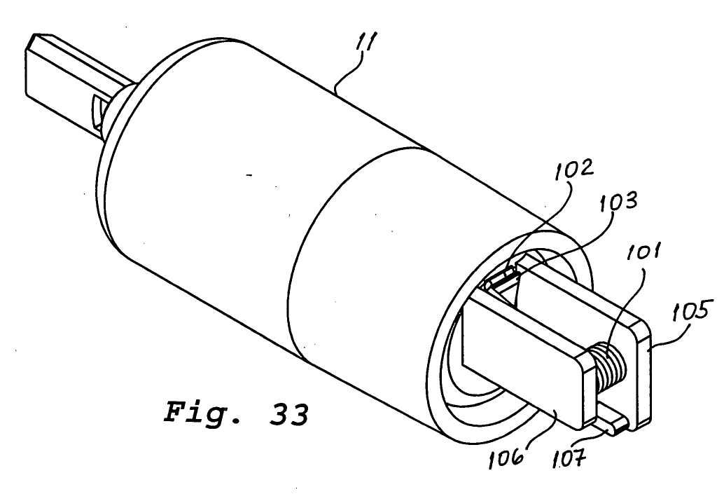

FIGS. 25 to 33 show a third embodiment of ignition means suitable for use as part of an injection device having a structure similar to the structure of the above described injection device 11 in FIG. 1. This third embodiment comprises an igniter 81 described with reference to FIGS. 25 to 30 and means for effecting a mechanical impact on a spot 82 of the outer surface of igniter 81. Spot 82 lies in a ignition impact zone. The means for effecting a mechanical impact on spot 82 are described below with reference to FIGS. 31 to 33.

Other examples of structures of injection devices are described hereinafter in the description of other embodiments comprising different ignition means. The ignition means described below with reference to FIGS. 25 to 33 are also suitable for those other examples of structures of injection devices.

FIG. 25 shows a partial view of an injection device having a similar structure as injection device 11 shown in FIG. 1, but wherein an igniter 81 described hereinafter replaces the firing pin 5 and the impact sensitive primer material 2 shown in FIG. 1 as ignition means.

Igniter 81 is inserted and held in a suitable opening of an ignition plate 62 that is a gas-tight closure of the rear part of a propellant chamber 63.

Igniter 81 comprises a first body, e.g. a metal tube 84, which is deformable, can be bent, and the interior of which is a cavity which is fluidically connected with propellant chamber 63.

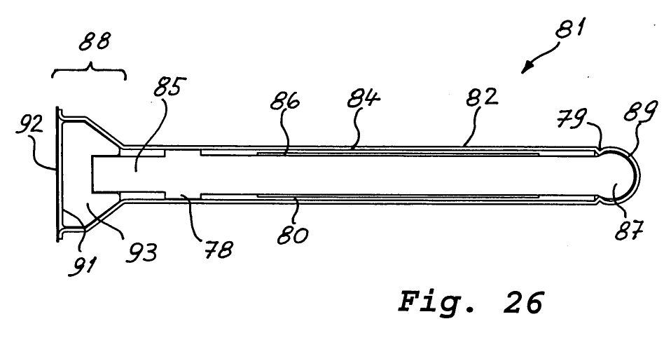

As can be appreciated from FIG. 26 which shows the structure of igniter 81 more in detail, a second body, e.g. a metallic anvil pin 85 is arranged within metal tube 84 so that a portion of the outer surface of anvil pin 85 is spaced from and is in face of a portion of the inner surface of tube 84. There is an air gap 80 between the latter surface portions. An end part 87 of pin 85 is retained within the closed end part of tube 84 by a retaining crimp 79. Centering lugs 78 at the opposite end of pin 85 ensure the spacing between the above mentioned surface portions.

A middle zone of the outer surface of anvil pin 85 is coated with an impact sensitive pyrotechnic material 86.

In a preferred embodiment one of the above-mentioned surface portions which face each other is coated with an impact sensitive pyrotechnic material and the other is coated with a secondary ignition material.

In another preferred embodiment one or both of the above-mentioned surface portions which face each other is coated with a layer of an impact sensitive pyrotechnic material and a layer of a secondary ignition material.

An impact applied on spot 82 of the outer surface of tube 84 by mechanical means described hereinafter causes a corresponding impact on impact sensitive pyrotechnic material 86 and thereby ignites this material. The products of this ignition ignite a propellant contained in propellant chamber 63.

As shown by FIG. 26, tube 84 has a closed end and an open, funnel shaped end portion 88 that ends in an outlet 91 open towards the propellant chamber 63. Tube 84 has a retaining crimp 79 that keeps anvil pin 85 in a predetermined axial position within tube 84.

In a preferred embodiment outlet 91 is sealed with a frangible moisture seal 92 that excludes penetration of water vapor into the interior of tube 84 prior to use of igniter 81.

In a further preferred embodiment, a secondary ignition material 93, e.g. a primer mix, is provided and located outside of tube 84 and near the outlet 91 of tube 84, so that products of combustion of the impact sensitive pyrotechnic material 86 ignite the secondary ignition material and thereby increase the amount of hot material for igniting the propellant in propellant chamber 63.

In another further preferred embodiment, a secondary ignition material 93, e.g. a primer mix, is provided and located inside of tube 84 and near the outlet 91 of tube 84, so that products of combustion of the impact sensitive pyrotechnic material 86 ignite the secondary ignition material 93 and thereby increase the amount of hot material for igniting the propellant in propellant chamber 63.

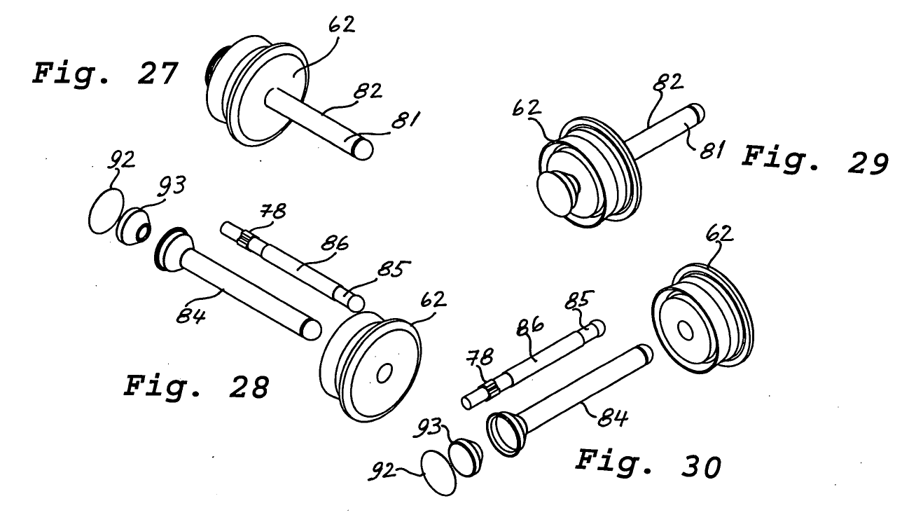

FIG. 27 shows a perspective view of igniter 81 inserted in ignition plate 62. FIG. 28 shows an exploded perspective view of the assembled components shown by FIG. 27.

FIGS. 29 and 30 are similar to FIGS. 27 and 28, but show views taken from a different point of view.

A simplified variant of the embodiment described above with reference to FIGS. 25 to 30, the device does not include metallic anvil pin 85. In this simplified embodiment a portion of the inner surface of tube 84 is coated with an impact sensitive pyrotechnic material. When a mechanical impact is effected, e.g. by means described hereinafter, on a spot of the outer surface of tube 84 opposite to the portion of the inner surface coated with the impact sensitive pyrotechnic material, the latter impact ignites the impact sensitive pyrotechnic material and the products of this ignition ignite the propellant.

A spring percussion striker mechanism for applying an impact to the outer surface of igniter 81 is described hereinafter with reference to FIGS. 31 to 33.

FIGS. 31 to 33 are perspective views of the rear of an injection device 11 of the type described above with reference to FIG. 1 and including an igniter 81 of the type described above with reference to FIGS. 25 to 30. FIG. 31 also shows a spring mechanism which is arranged for impacting on a spot of the outer surface of igniter 81 when a trigger releases a latch.

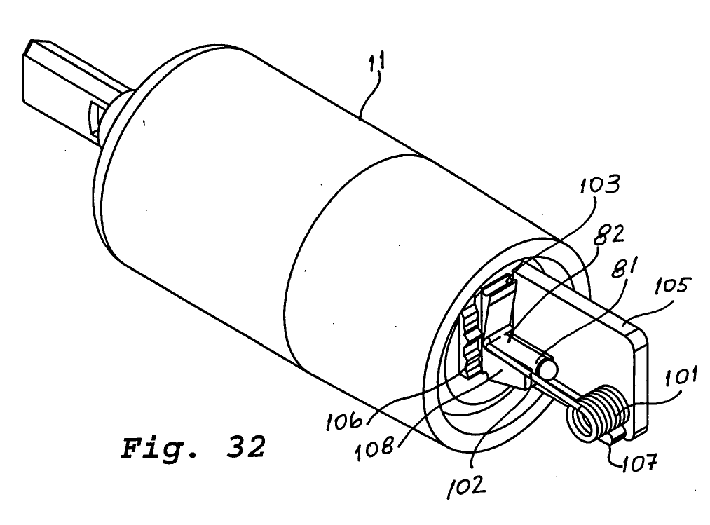

The spring striker comprises a torsion spring coil 101 with two L-shaped arms 102. A release latch 103 which makes possible to set the spring in a cocked state shown by FIG. 31. The spring striker further comprises two trigger guard plates 105 and 106 that prevent inadvertent actuation of torsion spring 101. In FIGS. 31 and 32 trigger guard 105 is cut away so that the torsion spring coil 101 and the impact-sensitive igniter 81 are visible.

The above described torsion spring coil is actuated by pushing the coil of the spring towards a limit stop 107 to pull the end of the upper L-arm of spring 101 out of the release latch 103 so that the L-arm it strikes the sensitive area of igniter 81 which is supported by a support 108.

Igniter tube 81 extends from the rear of the cartridge so that the impact sensitive area of the igniter, that is the portion thereof which lies above the pyrotechnic material contained in tube 84 of igniter 81 is exposed to impact from the spring striker. In order to increase the effect of the impact against the tube, igniter 81 is supported by support 108 below the point where the L-arm of torsion spring coil 101 impacts on it.

Torsion spring coil 101 is installed at the rear of the cartridge housing of injection device 11 by spreading the L-arms 102 and hooking them into two notches or latches, a release latch 103 and a pivot notch 104. Torsion spring coil 101 is preferably installed before igniter 81 so that a slip in the installation procedure will not result in accidental ignition.

The release notch 103 is shaped such that when the spring coil 101 is pushed down, the top L-arm 102 snaps out of the release notch 103, accelerates to high velocity, and strikes the side of igniter 81. The lower notch 104 acts as a simple pivot.

In use, the rear of the cartridge of injection device 11 is inserted into a disposable or reusable holder (not shown). The holder includes a lever or button that engages the torsion spring coil 101. When the user presses the lever or button, the motion actuates the torsion spring coil.

The above described spring percussion striker mechanism has the following advantages:

Torsion springs have high natural frequency, providing a high velocity, high-energy impact at acceptable stress levels.

The general class of spring required is low in cost and widely available.

The release latch, notch pivot and trigger guards are adapted to be manufactured by injections molding as part of the cartridge housing of injection device 11, and therefore only require a very little increase of the manufacturing cost.

Since a simple push is required to actuate the spring mechanism, a very simple holder mechanism is sufficient for the desired operation.

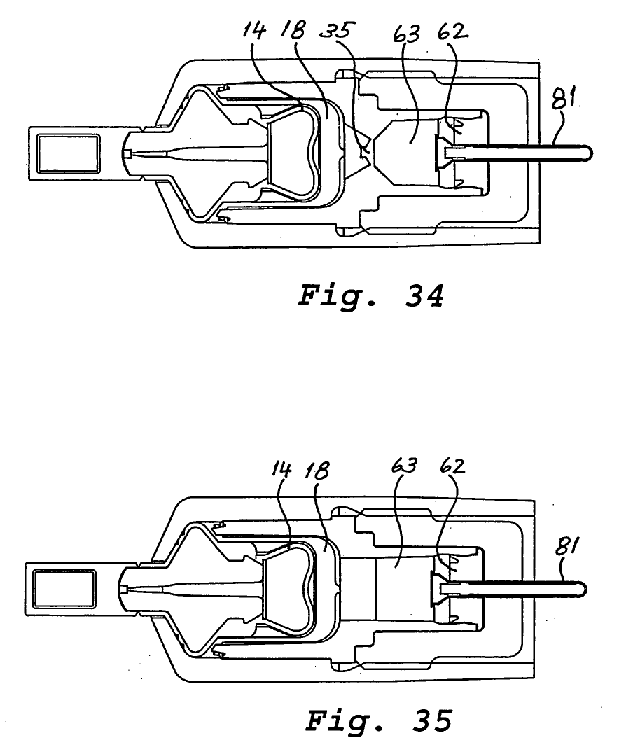

The ignition means described above with reference to FIGS. 25 to 33 can be used with various embodiments of needle free injection devices, e.g. with the embodiments shown by FIGS. 34 to 37. In these Figures each of the injection devices shown comprises an igniter 81 which has the above described structure. A spring percussion striker mechanism for applying an impact to the outer surface of igniter 81 as described above with reference to FIGS. 31 to 33 is also used in combination with each of these injections devices, but is not shown in FIGS. 34 to 37.

FIG. 34 shows an injection device which has the structure described in the description of Embodiment 1 with reference to FIGS. 1 to 3.

FIG. 35 shows an injection device which has a similar structure, but wherein there is no wall or membrane that separates the propellant chamber 63 from the zone of the injection device where the medication container is located, so that pressure generated within propellant chamber 63 is directly applied to deformable/elastic barrier 18 and thereby to the deformable wall 14 of the medication container 12.

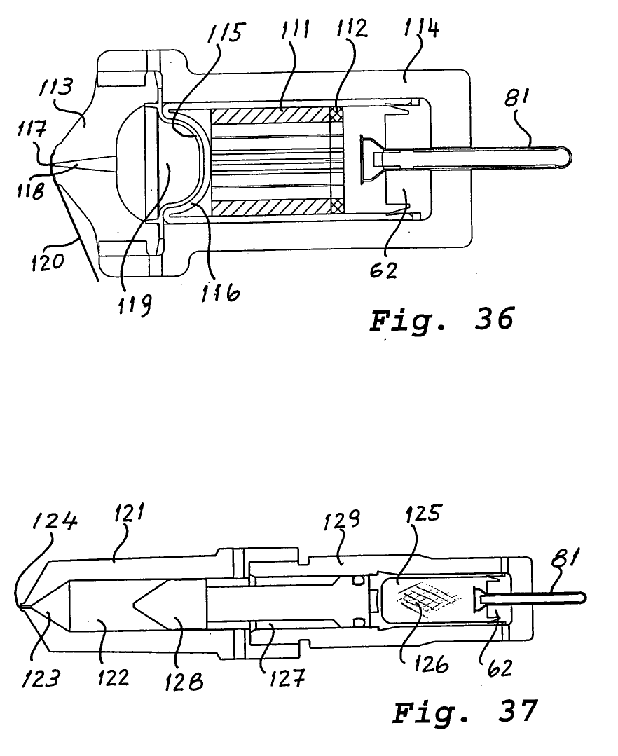

FIG. 36 shows an injection device wherein the propellant used is e.g. a one-piece propellant 111 provided with an ignition layer 112 located close to the outlet of igniter 81. The injection device shown by FIG. 36 comprises a nozzle body 113 and a rigid housing 114. Housing 114 has a first open end adapted to receive and be connected with the nozzle body and a second closed end. The interior of housing 14 defines a chamber which extends between the open end and the closed end of housing 114. This chamber is adapted to receive a first deformable diaphragm 115 which together with a cavity of said nozzle body forms a medication chamber 119 suitable for receiving a predetermined amount of a medication, and a second deformable diaphragm 116 a portion of which extends around a portion of said first deformable diaphragm 115. Second deformable diaphragm 116 and housing 114 form together a chamber for receiving a propellant and means for igniting said propellant. Nozzle body 113 has at its outer end an orifice 117 which is the outlet of a channel 118 for ejecting said medication out of chamber 119 when a gas pressure generated by ignition of said propellant 111 is applied to second deformable diaphragm 116 and thereby to said first deformable diaphragm 115. Before use orifice 117 is sealed by removable peel off foil seal tab 120.

In a preferred embodiment nozzle body 113 and rigid housing 114 are integrally built as a single-piece element having a rear opening and are made of one and the same material. The rear opening is closed by an ignition plate that holds suitable ignition components.

FIG. 37 shows an injection device comprising a rigid medication container 121 and a housing part 129 connectable thereto. The injection device shown by FIG. 37 comprises a medication zone 122 for receiving a liquid medication, a nozzle 123 which has an outlet orifice 124 and which is in fluidic communication with medication zone 122, a propellant zone 125 where a propellant 126 is located, a channel 127 that fluidically connects propellant zone 125 with medication zone 122, and a piston 128 slidably arranged within channel 127, so that upon ignition of propellant 126 gas pressure generated by combustion of the propellant exerts pressure on piston 128 and thereby causes a displacement of piston 128 which then exerts pressure on liquid medication contained in medication zone 122 and ejects it through the outlet orifice 124 of nozzle 123.

In a modification of the embodiment shown by FIG. 37, piston 128 is replaced e.g. by a piston having a front part of a first material suitable for contact with a medication and a rear part of a different second material suitable for contact with the hot gas generated by combustion of the propellant, or by a piston consisting of two separate parts which before the injection process are spaced from each other, e.g. a front part in contact with the medication and a rear part located close to the propellant. In the latter case, pressure generated by combustion of the propellant causes an accelerated motion of the rear part of the piston and an thereby an impact of this part on the front part of the piston which then exerts pressure on liquid medication contained in medication zone 122 and ejects it through the outlet orifice 124 of nozzle 123.

Embodiment 4

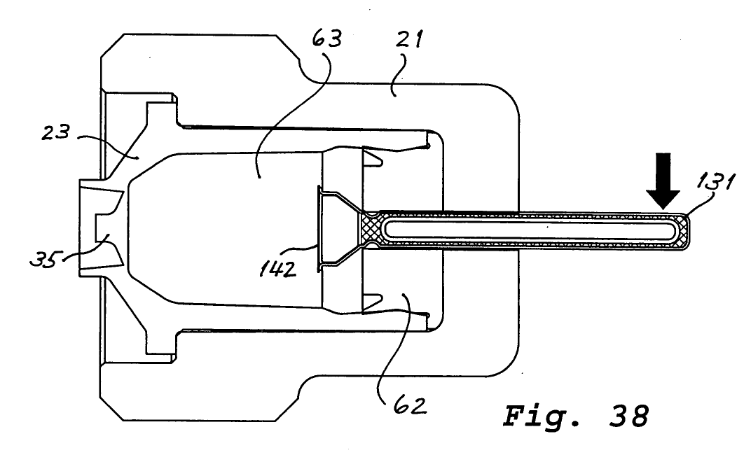

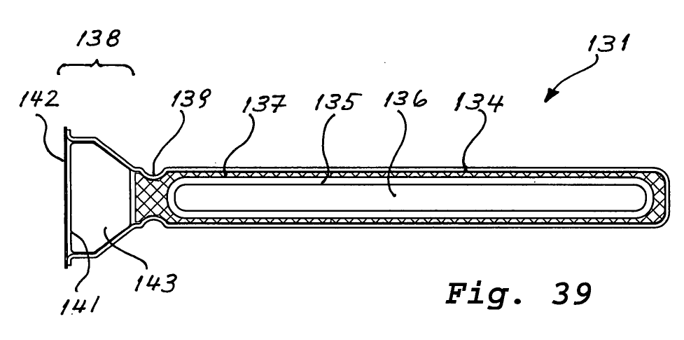

FIGS. 38 to 44 show an example of a fourth embodiment of ignition means suitable for use as part of an injection device having a structure similar to the structure of the above described injection device 11 in FIG. 1. FIGS. 45 to 50 show another example of the latter fourth embodiment of ignition means.

Other examples of structures of injection devices are described above and also hereinafter in the description of other embodiments comprising different ignition means. The ignition means described below with reference to FIGS. 38 to 44 are also suitable for those other examples of structures of injection devices.

FIG. 38 shows a partial view of an injection device having a similar structure as injection device 11 shown in FIG. 1, but wherein an igniter 131 described hereinafter replaces the firing pin 5 and the impact sensitive primer material 2 shown in FIG. 1 as ignition means.

Igniter 131 is inserted and held in a suitable opening of an ignition plate 62 that is a gas-tight closure of the rear part of a propellant chamber 63.

As can be appreciated from FIG. 39 which shows the structure of igniter 131 more in detail, igniter 131 comprises a first body, e.g. a metal tube 134, which is deformable, can be bent, and the interior of which is a cavity which is fluidically connected with propellant chamber 63. Igniter 131 further comprises a second tube 135 which is closed at both ends and arranged within tube 134 so that the outer surface of second tube 135 is spaced from the inner surface of first tube 134. Second tube 135 is e.g. a breakable glass ampoule which is adapted to be broken by bending of first tube 134 when that bending exceeds a predetermined threshold value. Second tube 135 contains a first member 136 of a hypergolic pair. First member 136 is e.g. a hypergolic fluid, i.e. a liquid or a gas.

A second member 137 of a hypergolic pair is contained in a space between the inner surface of first tube 134 and the outer surface of second tube 135. Second member 137 is e.g. a hypergolic powder.

Certain chemical pairs are hypergolic, and will ignite and burn spontaneously when mixed. Examples include:

Sulfuric acid (liquid) added to a mixture of potassium chlorite and sugar (powder).

Triethyl aluminum (volatile liquid) added to air.

Glycerin (liquid) added to potassium permanganate (powder).

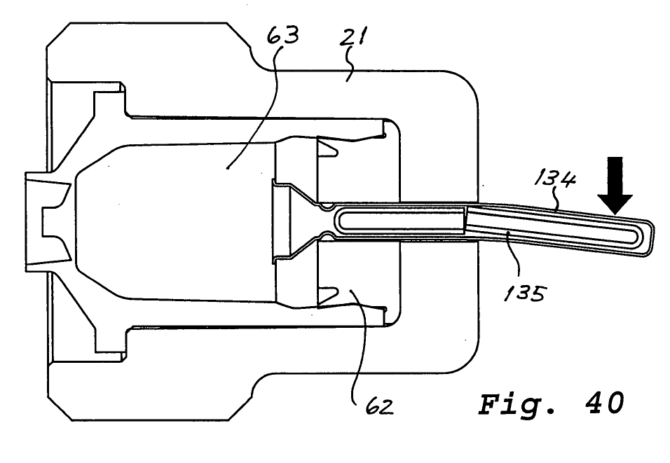

FIG. 40 shows that when first tube 134 is bent beyond a threshold value, second tube 135 is broken by that bending of first tube 134. When this happens, the first member 136 and the second member 137 of the hypergolic pair contact each other and their chemical reaction causes an ignition and the products of this ignition ignite the propellant contained in propellant chamber 63.—Igniter 131 is actuated by bending tube 134 to one side using a push button or similar means.

As shown by FIG. 39, tube 134 has a closed end and an open, funnel shaped end portion 138 that ends in an outlet 141 open towards the propellant chamber 63. Tube 134 has a retaining crimp 139 that keeps tube 135 and second hypergolic member 137 in a predetermined axial position within tube 134. Tube 135 is further secured in place by dimples formed in the wall of tube 134,

In a preferred embodiment outlet 141 is sealed with a frangible moisture seal 142 that excludes penetration of water vapor into the interior of tube 134 prior to use of igniter 131.

In a further preferred embodiment, a secondary ignition material 143, e.g. a primer mix, is provided and located outside of tube 134 and near the outlet 141 of tube 134, so that products of ignition of the hypergolic members ignite the secondary ignition material and thereby increase the amount of hot material for igniting the propellant in propellant chamber 63.

In another further preferred embodiment, a secondary ignition material 143, e.g. a primer mix, is provided and located inside of tube 134 and near the outlet 141 of tube 134, so that products of ignition of the hypergolic members ignite the secondary ignition material 143 and thereby increase the amount of hot material for igniting the propellant in propellant chamber 63.

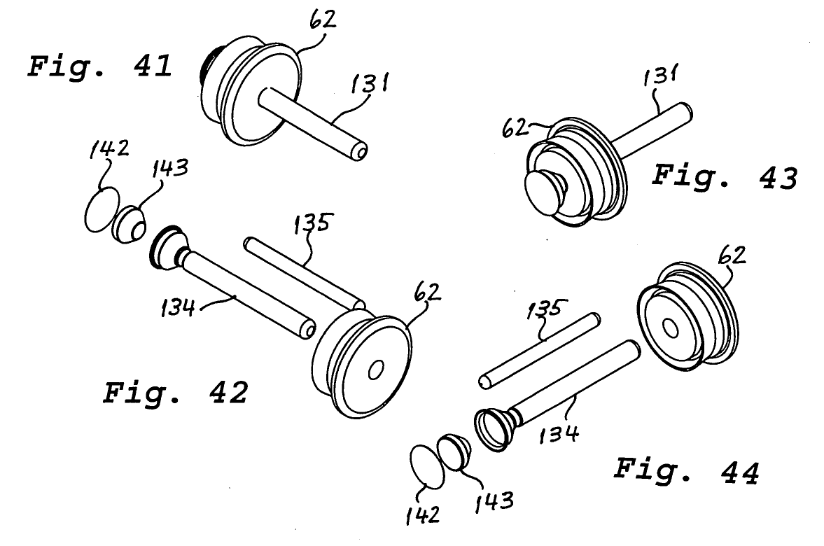

FIG. 41 shows a perspective view of igniter 131 inserted in ignition plate 62. FIG. 42 shows an exploded perspective view of the assembled components shown by FIG. 41.

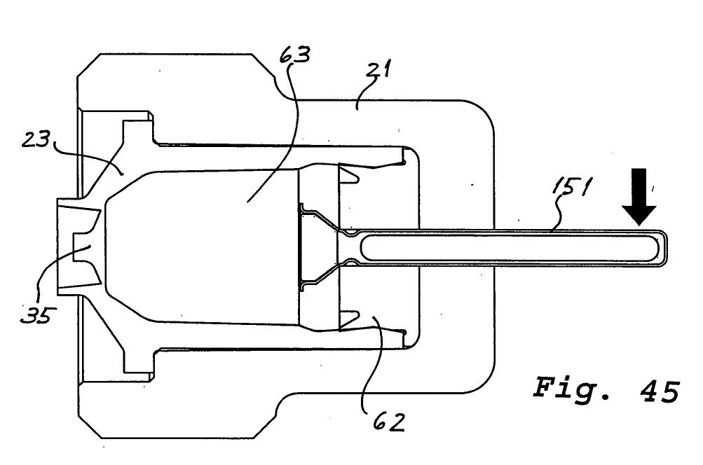

FIGS. 43 and 44 are similar to FIGS. 41 and 42, but show views taken from a different point of view.

FIG. 45 shows a partial view of an injection device having a similar structure as injection device shown in FIG. 38, but in this embodiment an igniter 151 having a different structure is inserted an held by ignition plate 62.

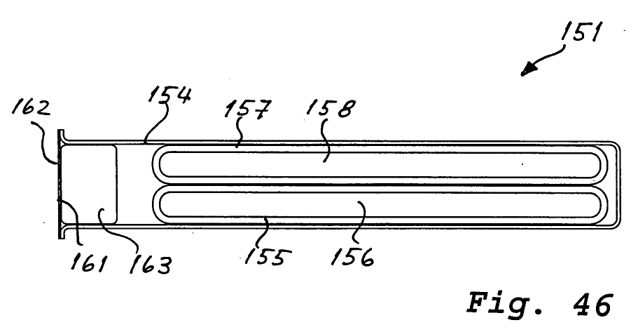

As shown by FIG. 46, igniter 151 comprises an elongated container 154 which has a closed end and an open end through which the interior of container 154 is fluidically connected with a zone within the device where a propellant is located. Container 154 is deformable and can be bent.

Container 154 contains a first tube 155 and a second tube 157. Both tubes 155, 157 are closed at both ends and arranged within container 154. First tube 155 contains a first fluid member 156 of a hypergolic pair and second tube 157 contains a second fluid member 158 of a hypergolic pair.

Each of tubes 155, 157 is e.g. a breakable glass ampoule which is adapted to be broken by bending of first tube 134 when that bending exceeds a predetermined threshold value. Closed tubes 155 and 157 provide excellent storage conditions for the hypergolic members 156 and 158 respectively.

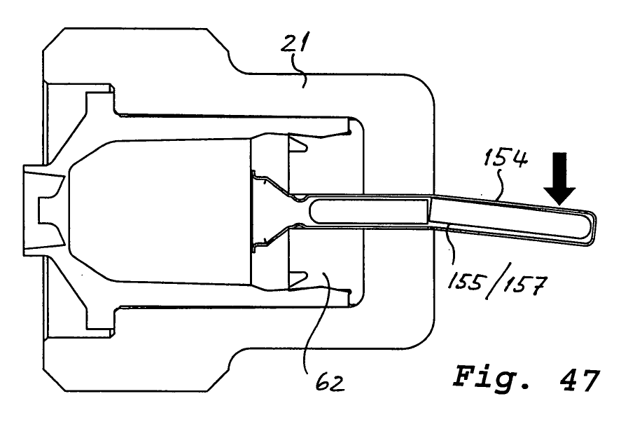

FIG. 47 shows that when container 154 is bent beyond a threshold value, both tubes 155, 157 are broken by that bending of container 154. When this happens, the first member 156 and the second member 158 of the hypergolic pair contact each other and their chemical reaction causes an ignition and the products of this ignition ignite the propellant contained in propellant chamber 63.

In a preferred embodiment, a secondary ignition material (not shown), is provided and located near the breaking points of tubes/ampoules 155, 157, so that products of combustion of the hypergolic pair ignite that secondary ignition material and thereby increase the amount of hot material for igniting the propellant.

In a further preferred embodiment, a secondary ignition material, e.g. a primer mix, is provided and located outside of container 154 and near the outlet 161 of container 154, so that products of ignition of the hypergolic members ignite the secondary ignition material and thereby increase the amount of hot material for igniting the propellant in propellant chamber 63.

In another preferred embodiment, a secondary ignition material 163, e.g. a primer mix, is provided and located inside of container 154 and near the outlet 161 of container 154, so that products of ignition of the hypergolic members ignite the secondary ignition material 163 and thereby increase the amount of hot material for igniting the propellant in propellant chamber 63.

In a further preferred embodiment outlet 161 is sealed with a frangible moisture seal 162 that excludes penetration of water vapor into the interior of container 154 prior to use of igniter 151.

FIG. 48 shows a perspective view of igniter 151 inserted in ignition plate 62.

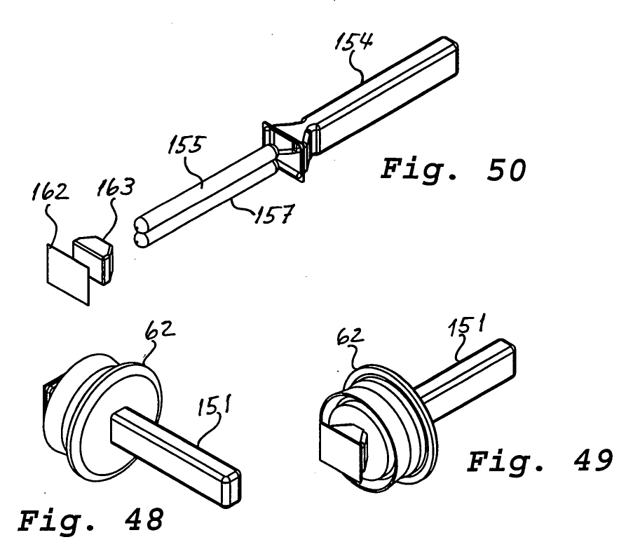

FIG. 49 is similar to FIG. 48, but show a view taken from a different point of view.

FIG. 50 shows an exploded perspective view of the assembled components shown by FIGS. 48 and 49 respectively.

Embodiment 5

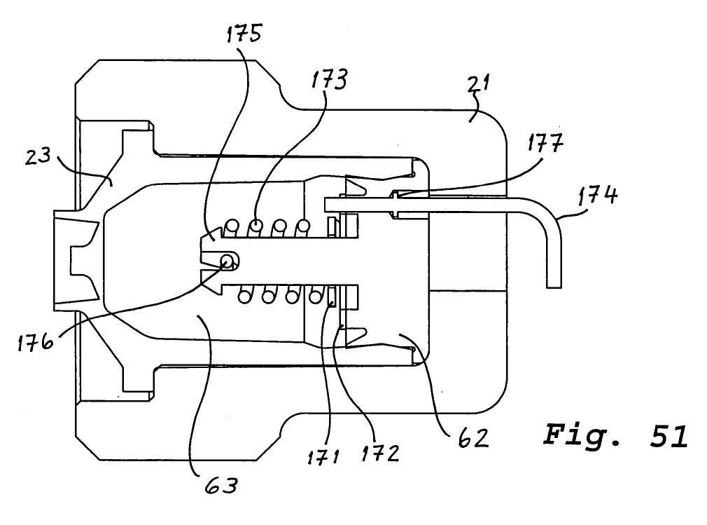

FIGS. 51 to 55 show an example of a fifth embodiment of ignition means suitable for use as part of an injection device having a structure similar to the structure of the above described injection device 11 in FIG. 1.

Other examples of structures of injection devices are described above and also hereinafter in the description of other embodiments comprising different ignition means. The ignition means described below with reference to FIGS. 51 to 55 are also suitable for those other examples of structures of injection devices.

FIG. 51 shows a partial view of an injection device having a similar structure as injection device 11 shown in FIG. 1, but wherein an igniter arrangement described hereinafter replaces the firing pin 5 and the impact sensitive primer material 2 shown in FIG. 1 as ignition means. The latter igniter arrangement makes use of safety match chemistry.

The igniter arrangement represented in FIG. 51 comprises a rotatable body 171, a stationary body 172, pressing means 173 for pressing at least a portion of rotatable body 171 against stationary body 172, a locking pin 174 that when locked prevents rotation of rotatable body 171, and driving means 175 for rotating rotatable body 171 when locking pin 174 is unlocked. At least a part of a portion of the outer surface of rotatable body 171 or at least a part of the outer surface of stationary body 172 is coated with a combustible mixture that bursts into flame when activated by friction. Pressing means bring a portion of the outer surface of rotatable body 171 in contact with stationary body 172.

When locking pin 174 is unlocked, driving means 175 cause a rotation of rotatable body 171. Under the pressing effect exerted by pressing means 173, such a rotation causes a friction of said combustible mixture against an outer surface of said stationary body 172 and thereby ignition of said mixture, the products of this ignition ignite a propellant contained in propellant chamber 63.

Rotatable body 171 is e.g. a two-armed match that is free to rotate on an anchor post 175 molded as part of ignition plate 62. The tips of these arms are coated with match head material that includes oxidizers, fuels and binders.

Stationary body 172 is e.g. a stationary striker ring coated with a fuel, e.g. red phosphorous and binders.

A combination of torsion and compression spring 173 is an example of means that are used to perform the function of pressing means and driving means. Such a spring loads rotatable match 171 against the striker ring 172, and is wound up so that it applies a torque to rotatable match 171.

The opposite end 176 of spring 173 is locked to the end of the anchor post 175.

Locking means are e.g. a formed wire actuation pull pin 174 which is pressed into a hole in ignition plate 62, and engages a lug on the rotary match 171 that prevents it from being rotated by the torque applied by spring 173.

Ignition plate 62 and the above described igniter arrangement closes the rear of the propellant container 23.

Rotatable match 171 and spring 173 are in propellant chamber 63 where a propellant is located, and actuation pull pin 174 extends through a slot in housing part 21. A flange 177 on pull pin 174 acts as a stop to limit the outward motion of this pin.

When pin 174 is pulled, spring 173 rotates the rotatable match heads 171 against the striker ring 172. A chemical reaction between the fuel, e.g. red phosphorous, on striker ring 172 and an oxidizer, e.g. the match head mixture on rotatable match 171 or vice-versa causes the match heads to ignite and flare, and this in turn ignites the propellant in propellant chamber 63.

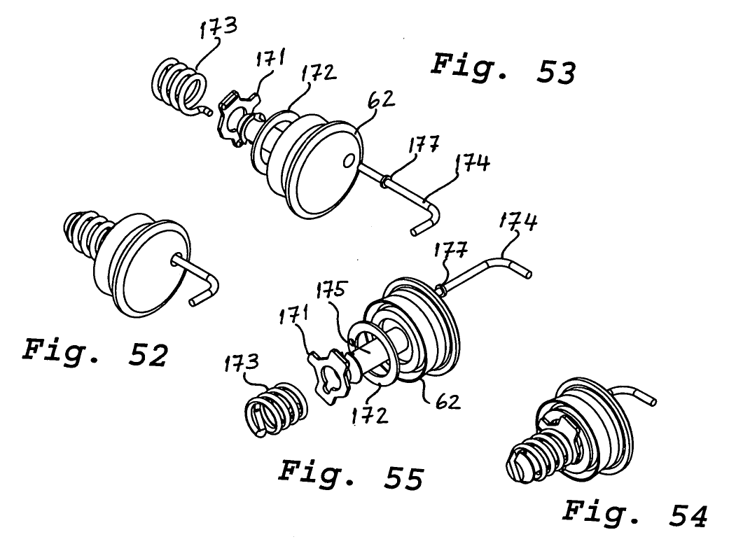

FIG. 52 shows a perspective view of the above described igniter arrangement assembled with ignition plate 62.

FIG. 53 shows an exploded perspective view of the components shown by FIG. 52.

FIGS. 54 and 55 are similar to FIGS. 52 and 53, but show views taken from a different point of view.

In a preferred embodiment the rotatable body 171 and the stationary body 172 are in a zone closed by a frangible closure (not shown) that protects coatings on those bodies against effects of humidity.

In another preferred embodiment a secondary ignition material (not shown) is provided and located near to the zone where the rotatable body 171 and the stationary body 172 contact each other.

Embodiment 6

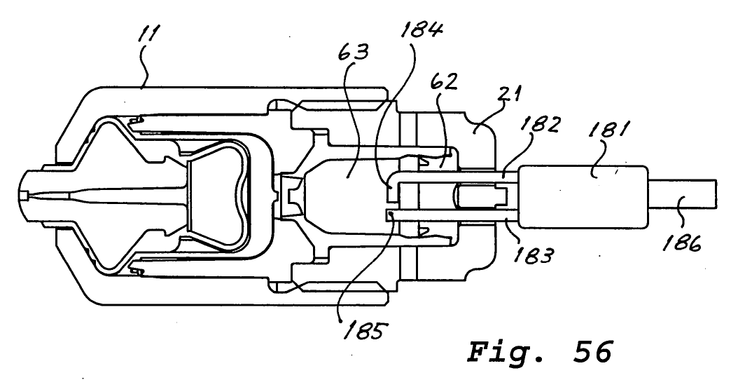

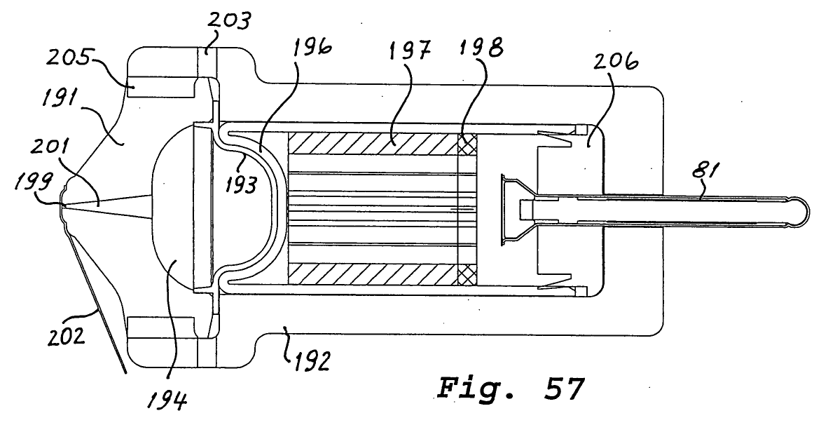

FIG. 56 shows an example of a sixth embodiment of ignition means suitable for use as part of an injection device having a structure similar to the structure of the above described injection device 11 in FIG. 1.

FIG. 56 shows a partial view of an injection device having a similar structure as injection device 11 shown in FIG. 1, but wherein an igniter arrangement described hereinafter replaces the firing pin 5 and the impact sensitive primer material 2 shown in FIG. 1 as ignition means.

As shown by FIG. 56 a piezoelectric spark generator 181 is arranged so that its electrodes 182, 183 pass through and are inserted in suitable bores in housing part 21 and ignition plate 62 and the spark generating end parts 184, 185 of the electrodes are located within propellant chamber 63, whereas the body of the piezoelectric spark generator 181 and the actuation push button 186 thereof are outside the housing part 21 and outside the injection device 11.

Small piezoelectric ignition modules that generate electric sparks with voltage on the order of some thousands of volts are widely used as igniters in disposable cigarette lighters. They are operated by a simple pushing action. Their simplicity, cost and reliability make them suitable for ignition of needle free injection devices having any of the above described structures.

The ignition means described above with reference to FIG. 56 are also suitable for other examples of structures of injection devices described above in the description of other embodiments comprising different ignition means.

7) Use of One or More One-Piece Propellant Forms in Combination with Any of the Above-Described Non-Electric Ignition Means

FIG. 57 shows an example of an injection device wherein the propellant 197 has the form of a one-piece pellet.

The injection device shown by FIG. 57 comprises a nozzle body 191 and a rigid housing 192 made of a plastic material. Housing 192 has a first open end adapted to receive and be connected with the nozzle body 191 and a second closed end.

The interior of the housing 192 defines a chamber which extends between the open end and the closed end of housing 192. That chamber is adapted to receive a first deformable diaphragm 193 which together with a cavity 194 of nozzle body 191 forms a medication chamber 195 suitable for receiving a predetermined amount of a medication, and a second deformable diaphragm 196 a portion of which extends around a portion of the first deformable diaphragm 193. The second deformable diaphragm 196 and the housing 192 form together a chamber for receiving a one-piece propellant pellet 197 and means for igniting this propellant.

Housing 192 further contains an ignition layer 198 which is in contact with or is an integral part of the one-piece propellant pellet 197. Ignition layer is located to an igniter 81 of the type described above with reference to FIGS. 25-33. Igniter 81 is held by an ignition plate 206.

Nozzle body 191 has at its outer end an orifice 199 which is the outlet of a channel 201 for ejecting the medication out of chamber 195 when a gas pressure generated by ignition of the propellant 197 is applied to the second deformable diaphragm 196 and thereby to the first deformable diaphragm 193.

Nozzle body 191 is made e.g. of polypropylene and the first deformable diaphragm 193 is made e.g. of polyethylene. Both polypropylene and polyethylene are materials suitable and accepted for long term storage of many medications.

In the example described with reference to FIG. 22, the amount of medication stored in medication container 194, 195 is e.g. 200 microliters.

An important characteristic of the injector structure shown by FIG. 57 is that both the medication container and the propellant are actually both contained in a single chamber. This structure minimizes heat losses and thereby minimizes the amount of energy and thereby the amount of propellant required to generate the gas pressure necessary for performing an injection. In the present example an amount of propellant corresponding to about 20 milligrams of a nitrocellulose based composition was used.

The orifice 199 of the nozzle body 191 is sealed by a removable foil seal 202.

In a preferred embodiment the housing 192 and the nozzle body 191 are connectable to each other by a screw connection 205.

In another preferred embodiment nozzle body 191 and rigid housing 192 are integrally built as a single-piece element having a rear opening and are made of one and the same material. The rear opening is closed by an ignition plate that holds suitable ignition components.

The first deformable diaphragm 193 and nozzle body 191 are clamped together by the screw connection 205 of housing 192 and nozzle body 191.

In another preferred embodiment the housing 192 has venting holes 203 located near to the outer edge of the first deformable diaphragm 193. In operation when propellant 197 is ignited and generates pressure, this pressure is applied to the second deformable diaphragm 196 and this diaphragm pressurizes the first deformable diaphragm and thereby the medication contained in medication container 194, 195 and causes fluid to flow into the nozzle channel 201 and be ejected as a jet through orifice 199. The space between the first diaphragm 193 and the second diaphragm 196 is vented by venting holes 203 to ensure that pressurized gas cannot enter into contact with the medication volume.

In a further preferred embodiment the housing 192 and the nozzle body 191 are so configured and dimensioned that they can withstand alone the pressure generated by ignition of the propellant 197.

Nozzle body 191 has preferably a tapered outer surface which has its smallest cross-section at the orifice 199 at the outer end of the nozzle body 191.

A simplified variant of the embodiment described above with reference to FIG. 57 does not include the second diaphragm 196, but only the first diaphragm 193.

### EXAMPLES OF PROPELLANT FORMS THAT CAN BE USED WITH ANY OF THE ABOVE DESCRIBED EMBODIMENTS OF A DEVICE ACCORDING TO THE INVENTION

A propellant form which can be used with any of the above described embodiments of a needleless hypodermic injection device is described hereinafter with reference to the injection device described above with reference to FIG. 57.

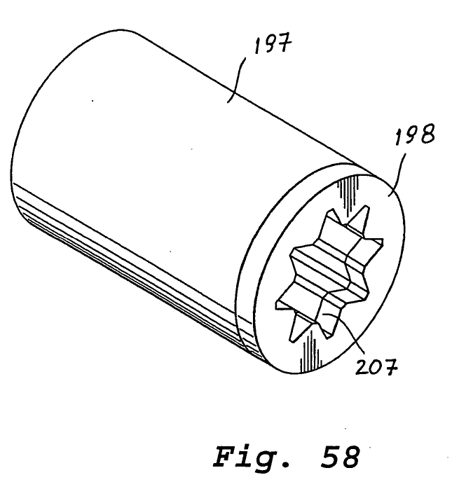

In the embodiment shown by FIG. 57, the propellant 197 is a one-piece propellant pellet. This pellet has e.g. the cylindrical or pillar shape shown by FIG. 58 and contains the main propellant charge for making an injection. The specific shape of the pellet can have features which allow to place it at a predetermined position within housing 192, e.g. for ensuring a good contact of the pellet with the outlet of igniter 81.

Within the scope of the invention a propellant pellet is a monolithic structure that contains one or more pre-measured pyrotechnic components. Such a pellet is handled and assembled in the gas generator as a discrete component. Use of such a propellant pellet thus eliminates the need to weigh-out and pour a propellant in powder or liquid form into a propellant container. A preferred embodiment of a propellant pellet of the kind just mentioned has zones having different properties in order to enhance the performance of the pellet. The pellet has e.g. the shape of a cylinder made of a nitrocellulose based composition and one end of this cylinder has an ignition mixture coating and this end of the cylinder is positioned next to an igniter.

Compared with prior art use of propellant in powder form, use of a one-piece propellant pellet offers the advantage of a simplification of the process for manufacturing the injection device, because the pellet comes to the process as a component having a specified weight which is simply inserted into the housing of the injection device, so that no weighing and filling machinery is necessary for handling the pellet. Propellant in powder form has on the contrary to be weighted as part of the manufacturing process and for this purpose weighing and filling machinery is necessary.

Pellets with a wide range of shapes and combinations of material are possible, providing flexibility in tailoring performance and fitting various physical configurations.

In a preferred embodiment, an ignition layer 198 is in contact with or is an integral part of the one-piece propellant pellet 197. Ignition layer 198 contributes to igniting propellant 197 and additionally provides the energy necessary for generating an initial fast rising pressure pulse.

As shown by FIG. 58, propellant pellet 197 preferably has e.g. a hole 207 which extends through pellet 197 and has a star-shaped cross-section that provides an increased surface area that contributes to a rapid ignition and which provides a gas flow passage through pellet 197.

The following are examples of the chemical and structural composition of a propellant pellet 197:

### Example A

Pellet 197 consists of only one grade cotton wool or guncotton which has been processed as a cord and which has a well defined weight per length. Cylindrical pellets 197 having predetermined dimensions and weight are obtained by cutting the cord in equidistant pieces. One end of each pellet so obtained has an ignition mixture coating. Defined positioning of the pellet into a gas generator will bring this coated end of the pellet close to an igniter.

### Example B

The base material of a pellet contains a defined mixture of two or more varieties of cotton wool or guncotton with different fiber length and reactivity. This material is felt and inserted under defined conditions (weight per length/volume) into a thin tube of polyethylene having an inner diameter of e.g. 0.1 mm. Cylindrical pellets 197 having predetermined dimensions and weight are obtained by cutting the tube into adequate cylindrical segments.

Each pellet so obtained is inserted into a gas generator and is arranged close to an igniter.

### Example C

A pellet of cotton wool or guncotton according to example A) or example B) is produced by a method wherein additional defined amounts of other materiel like capsules of liquid are included in the pellet.

### Example D

A first pellet of cotton wool or guncotton according to example A) or example B) is produced, but with a shorter length. A second pellet with different properties—with or without propellant properties—is set into a free space within the gas generator after having placed the first pellet within the gas generator.

The second pellet contains e.g. embedded salts, a filler (e.g. an aerogel) or capsules containing a liquid. A suitable filler is an aerogel. An aerogel is e.g. a fine powder that can be used as a filler in a mixture of other chemicals, e.g. in an ignition mixture.

The second pellet has a hole in its center (the second pellet has a toroid-like shape) and serves as a modifier of the burning behavior of the first pellet.

A one-piece propellant pellet 197 is so manufactured that the pellet or the method for its manufacture has one or more of the following features in order to achieve desired operation characteristics:

a) Propellant pellet 197 is manufactured from a selected material, e.g. a nitrocellulose based composition, or from a combination of selected materials.

b) Propellant pellet 197 is so manufactured that it has a specified shape and mass.

c) In the process for manufacturing propellant pellets suitable ignition materials can be integrated into the propellant pellet and located at selected spots, inside the pellet or on its outer surface.

d) In the process for manufacturing propellant pellets free space between the pellet and ignition means may be provided by choice of a suitable shape of the pellet. This free space may optionally be filled e.g. with powder or with a filamentary ignition material, e.g. guncotton.

e) The pellet is a mechanical assembly of components with different properties.

f) The pellet includes aggregates of soft filamentary material such as guncotton or capsules of liquid.

g) The pellet includes geometric features such as holes or ribs to increase surface area.

h) A pellet is a structure which fits alone or combined with other pellets properly into the inner space of a gas generator, thus avoiding unduly uncontrolled displacements thereof.

i) A part of the pellet (or an additional pellet) includes a region which only acts as a spacer without propellant properties and which serves for getting the total pellet system properly fitted into the gas generator.

j) The pellet has a self-supporting structure that keeps its shape, e.g. woven, plaited or felted filamentary material structure such as guncotton.

k) The pellet has an additional cover or envelope for stabilizing the structure of the pellet, e.g. a thin tube-like or net-like mantle of e.g. polyethylene or paper-like material.

Two or more one-piece propellant pellets 197 having the same or different characteristics can be arranged within housing 192 with or without intermediate materials between them instead of a single one-piece pellet in order to achieve particular effects like accelerating or delaying certain phases of the combustion of the propellant.

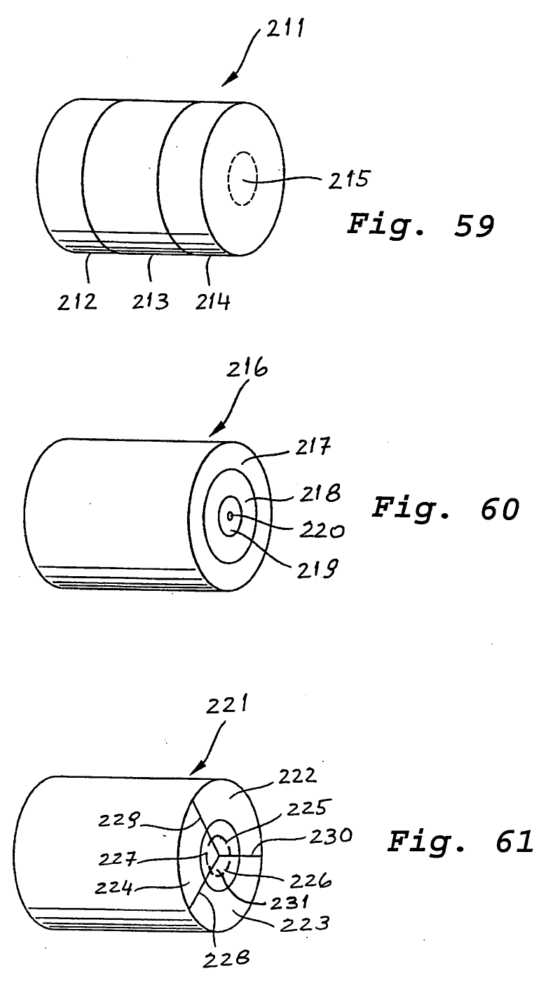

In preferred embodiments, the propellant 197 comprises an array of one-piece propellant pellets having each a predetermined shape, a predetermined chemical composition and a predetermined relative position within the array. Use of one-piece propellant pellets having different chemical compositions and therefore different burning properties make it possible to optimize the variation with time of the injection pressure generated by combustion of the propellant according to predefined criteria. FIGS. 59 to 61 show examples of such arrays.

FIG. 59 shows a stack 211 of cylindrical one-piece pellets 212, 213, 214. In a preferred embodiment, a hole 215 extends through the central portion of stack 211.

FIG. 60 shows an array 216 of concentric cylindrical one-piece pellets 217, 218, 219. In a preferred embodiment, a hole 220 extends axially through the central portion of array 216. In a preferred embodiment element 219 is left out to provide a larger hole axially extending through the central portion of array 216.

FIG. 61 shows an array 221 of one-piece pellets 222 to 227. Pellets 222 to 224 have each the shape of a segment of a cylinder having a predetermined wall thickness. Such segments are obtained by cutting a cylinder along planes parallel to the symmetry axis of the cylinder and passing through radii 228, 229, 230. Pellets 225 to 227 have each the shape of a segment of a rod having a predetermined diameter. Such segments are obtained by cutting a rod along planes parallel to the symmetry axis of the rod and passing through radii 228, 229, 230. In a preferred embodiment, a hole 231 axially extends through the central portion of array 221. In a preferred embodiment one or more of elements 225-227 is left out to provide a larger hole axially extending through the central portion of array 216.

In preferred embodiments of the examples shown by FIGS. 59 to 61, an ignition layer is in contact with or is an integral part of an array of one-piece propellant pellets.

Propellant pellets of the above described types preferably have a coating protecting them against deterioration caused by humidity or by abrasion; in particular abrasion caused by transport, handling or storage processes.

Although preferred embodiments of the invention have been described using specific terms, such description is for illustrative purposes only, and it is to be understood that changes and variations may be made without departing from the spirit or scope of the following claims.

1. A device for performing a needleless hypodermic injection of a liquid medication contained in the device, said device comprising pyrotechnical means for generating within the device a pressure necessary for injecting the medication, said device comprising ignition means for igniting a propellant contained in said device, said ignition means comprising
an impact initiated primer material and a firing pin for striking said primer material, said primer material being so positioned with respect to said propellant that when the firing pin strikes the primer material the hot products of combustion of said primer material ignite said propellant.

2. The device according to claim 1, wherein the impact initiated primer material is contained in a hollow rim of a shell case and the firing pin is adapted for crushing the rim at one point when actuated.

3. The device according to claim 1, wherein the impact initiated primer material is part of a centerfire primer arrangement consisting of a deformable cup, an anvil, and primer material, in which arrangement the firing pin indents the cup and crushes the primer material against the anvil when actuated.

4. The device according to claim 1, wherein ignition of the primer material alone produces sufficient gas volume and pressure that no additional propellant is needed for performing an injection with the device.

5. The device according to claim 1, wherein ignition of the primer material produces a first, fast high pressure pulse that starts the injection, and at the same time ignites a slower burning propellant that provides over a longer duration a lower pressure for completing the injection.

6. The device according to claim 1, wherein said primer material is contained in a container sealed with a frangible closure that excludes contact of the primer material with water vapor prior to use of the device.

7. The device according to claim 2, wherein the open end of the shell case is sealed with a frangible closure that excludes penetration of water vapor into the shell case prior to use of the device.

8. The device according to claim 2, wherein at least a portion of the propellant is contained within the shell case.

9. The device according to claim 7, wherein a portion of the propellant within the shell case has properties which are different from the properties of a portion of the propellant outside the shell case.

10. The device according to claim 3, wherein the primer material is sealed with a frangible closure that excludes contact of the primer material with water vapor prior to use of the device.

11. The device according to claim 1, said device further comprising
(a) a housing,
(b) a first chamber within said housing, said first chamber containing a medication unit configured and dimensioned to store a volume of liquid medication to be injected, said medication unit having a first region and a second region that are in liquid communication with each other, said first region being deformable and said second region having an ejection outlet, and
(c) a second chamber within said housing, said second chamber containing a propellant,
said first chamber comprising two zones, a first zone containing said medication unit and a second zone which is in communication with said second chamber, so that upon ignition of the propellant in the second chamber gas generated thereby expands into said second zone of said first chamber, exerts pressure on and deforms said deformable first region of said medication unit and thereby causes ejection of said liquid medication through said ejection outlet.

12. The device according to claim 11, wherein said propellant is contained in a propellant chamber having a wall which has a zone of reduced thickness which upon ignition of said propellant bursts and thereby forms an opening of said wall when gas pressure within the propellant chamber exceeds a predetermined value.

13. The device according to claim 1, said device further comprising
(a) a nozzle body, and
(b) a rigid housing,
said housing having a first open end adapted to receive and be connected with the nozzle body and a second closed end,
the interior of said housing defining a chamber which extends between said open end and said closed end of the housing, said chamber being adapted to receive
a first deformable diaphragm which together with a cavity of said nozzle body forms a medication chamber suitable for receiving a predetermined amount of a medication, and
a second deformable diaphragm a portion of which extends around a portion of said first deformable diaphragm,
said second deformable diaphragm and said housing forming together a chamber for receiving a propellant and means for igniting said propellant, and
said nozzle body having at its outer end an orifice which is the outlet of a channel for ejecting said medication out of said chamber when a gas pressure generated by ignition of said propellant is applied to said second deformable diaphragm and thereby to said first deformable diaphragm.

14. The device according to claim 13, wherein said nozzle body and said housing are integrally built as a single-piece element and are made of one and the same material.

15. The device according to claim 13, further comprising venting means for venting of the space comprised between the first deformable diaphragm and the second deformable diaphragm.

16. The device according to claim 13, without said second deformable diaphragm.

17. The device according to claim 1, said device further comprising
(a) a rigid medication container having a medication zone for receiving said liquid medication,
(b) a nozzle in fluidic communication with said medication zone, said nozzle having an outlet orifice,
(c) a propellant zone where said propellant is located within said device,
(d) a channel that fluidically connects said propellant zone with said medication zone, and
(e) piston means slidably arranged within said channel, so that upon ignition of said propellant gas pressure generated by combustion of the propellant causes displacement of said piston means which then exert pressure on said liquid medication and eject it through the outlet orifice of said nozzle.

18. A device for performing a needleless hypodermic injection of a liquid medication contained the device, said device including pyrotechnical means for generating within the device a pressure necessary for injecting the medication, said device comprising ignition means for igniting a propellant contained in said device, said ignition means comprising
(a) a first body having a cavity, said cavity having an outlet through which the interior of the cavity is fluidically connected with a zone within the device where said propellant is located, said first body being deformable by bending it,
(b) a second body arranged within said cavity so that a portion of the outer surface of the second body is spaced from a portion of the inner surface of said first body, said second body being to some extent elastically deformable by bending it, but adapted to be broken when that bending exceeds a predetermined threshold value, and
(c) said portion of the outer surface of the second body and said portion of the inner surface of said first body facing each other and one or both of those surface portions being coated with an impact sensitive pyrotechnic material, whereby an impact of said surface portions on each other caused by breaking the second body by bending of the first body and thereby bending of the second body beyond said threshold value ignites said impact sensitive pyrotechnic material and the products of this ignition ignite the propellant.

19. A device for performing a needleless hypodermic injection of a liquid medication contained in the device, said device including pyrotechnical means for generating within the device a pressure necessary for injecting the medication, said device comprising ignition means for igniting a propellant contained in said device, said ignition means comprising
(a) a tube having a closed end and an open end through which the interior of the tube is fluidically connected with a zone within the device where said propellant is located, said tube being deformable by bending it, and
(b) a breakable rod arranged within said tube so that a portion of the outer surface of the rod is spaced from a portion of the inner surface of said tube, said rod being to some extent elastically deformable by bending it, but adapted to be broken when that bending exceeds a predetermined threshold value,
said portion of the outer surface of the rod and said portion of the inner surface of said tube facing each other and one or both of those surface portions being coated with an impact sensitive pyrotechnic material, whereby an impact of said surface portions on each other caused by breaking the rod by bending of the tube containing the rod beyond said threshold value ignites said impact sensitive pyrotechnic material and the products of this ignition ignite the propellant.

20. The device according to claim 18, wherein the outlet of the cavity is sealed with a frangible closure that excludes penetration of water vapor into said cavity prior to use of the device.

21. The device according to claim 19, wherein the open end of said tube is sealed with a frangible closure that excludes penetration of water vapor into the interior of said tube prior to use of the device.

22. The device according to claim 18, wherein a secondary ignition material is provided and located outside of said cavity and near the outlet of the cavity, so that products of combustion of the impact sensitive pyrotechnic material ignite the secondary ignition material and thereby increase the amount of hot material for igniting the propellant.

23. The device according to claim 18, wherein a secondary ignition material is provided and located inside of said cavity and near the outlet of the cavity, so that products of combustion of the impact sensitive pyrotechnic material ignite the secondary ignition material and thereby increase the amount of hot material for igniting the propellant.

24. The device according to claim 18, wherein one of those surface portions which face each other is coated with an impact sensitive pyrotechnic material and the other is coated with a secondary ignition material.

25. The device according to claim 18, wherein one or both of those surface portions which face each other is coated with a layer of an impact sensitive pyrotechnic material and a layer of a secondary ignition material.

26. The device according to claim 19, wherein a secondary ignition material is provided and located outside of said tube and near the outlet of said tube, so that products of combustion of the impact sensitive pyrotechnic material ignite the secondary ignition material and thereby increase the amount of hot material for igniting the propellant.

27. The device according to claim 19, wherein a secondary ignition material is provided and located 7inside of said tube and near the outlet of said tube, so that products of combustion of the impact sensitive pyrotechnic material ignite the secondary ignition material and thereby increase the amount of hot material for igniting the propellant.

28. The device according to claim 19, wherein one of those surface portions which face each other is coated with an impact sensitive pyrotechnic material and the other is coated with a secondary ignition material.

29. The device according to claim 19, wherein one or both of those surface portions which face each other is coated with a layer of an impact sensitive pyrotechnic material and a layer of a secondary ignition material.

30. The device according to claim 18, said device further comprising
(a) a housing,
(b) a first chamber within said housing, said first chamber containing a medication unit configured and dimensioned to store a volume of liquid medication to be injected, said medication unit having a first region and a second region that are in liquid communication with each other, said first region being deformable and said second region having an ejection outlet, and
(c) a second chamber within said housing, said second chamber containing a propellant,
said first chamber comprising two zones, a first zone containing said medication unit and a second zone which is in communication with said second chamber, so that upon ignition of the propellant in the second chamber gas generated thereby expands into said second zone of said first chamber, exerts pressure on and deforms said deformable first region of said medication unit and thereby causes ejection of said liquid medication through said ejection outlet.

31. The device according to claim 30, wherein said propellant is contained in a propellant chamber having a wall which has a zone of reduced thickness which upon ignition of said propellant bursts and thereby forms an opening of said wall when gas pressure within the propellant chamber exceeds a predetermined value.

32. The device according to claim 18, said device further comprising
(a) a nozzle body, and
(b) a rigid housing,
said housing having
a first open end adapted to receive and be connected with the nozzle body and a second closed end,
the interior of said housing defining a chamber which extends between said open end and said dosed end of the housing, said chamber being adapted to receive
a first deformable diaphragm which together with a cavity of said nozzle body forms a medication chamber suitable for receiving a predetermined amount of a medication, and
a second deformable diaphragm a portion of which extends around a portion of said first deformable diaphragm,
said second deformable diaphragm and said housing forming together a chamber for receiving a propellant and means for igniting said propellant,
said nozzle body having at its outer end an orifice which is the outlet of a channel for ejecting said medication out of said chamber when a gas pressure generated by ignition of said propellant is applied to said second deformable diaphragm and thereby to said first deformable diaphragm.

33. The device according to claim 32, wherein said nozzle body and said housing are integrally built as a single-piece element and are made of one and the same material.

34. The device according to claim 32, further comprising venting means for venting of the space comprised between the first deformable diaphragm and the second deformable diaphragm.

35. The device according to claim 32, without said second deformable diaphragm.

36. The device according to claim 18, said device further comprising
(a) a rigid medication container having a medication zone for receiving said liquid medication,
(b) a nozzle in fluidic communication with said medication zone, said nozzle having an outlet orifice,
(c) a propellant zone where said propellant is located within said device,
(d) a channel that fluidically connects said propellant zone with said medication zone, and
(e) piston means slidably arranged within said channel, so that upon ignition of said propellant gas pressure generated by combustion of the propellant causes displacement of said piston means which then exert pressure on said liquid medication and eject it through the outlet orifice of said nozzle.

37. A device for performing a needleless hypodermic injection of a liquid medication contained in a medication unit within the device, said device including pyrotechnical means for generating within the device a pressure necessary for injecting the medication, said device comprising ignition means for igniting a propellant contained in said device, said ignition means comprising
(a) a first body having a cavity, said cavity having an outlet through which the interior of the cavity is fluidically connected with a zone within the device where said propellant is located, the tubular portion of said first body being deformable by a localized impact from one side,
(b) a solid anvil arranged within said tube so that a portion of the outer surface of the anvil is spaced from a portion of the inner surface of said tube, and
(c) means for effecting a mechanical impact on a spot of the outer surface of said first body, said portion of the outer surface of the anvil and said portion of the inner surface of said tube facing each other and one or both of those surface portions being coated with an impact sensitive pyrotechnic material, whereby an impact of said surface portions on each other effected by said means for effecting a mechanical impact ignites said pyrotechnic material and the products of this ignition ignite the propellant.

38. A device for performing a needleless hypodermic injection of a liquid medication contained in a medication unit within the device, said device including pyrotechnical means for generating within the device a pressure necessary for injecting the medication, said device comprising
a) a zone within the device where a propellant is located, and
b) ignition means for igniting said propellant, said ignition means comprising
(b.1) a hollow body having a cavity, said cavity having an outlet through which the interior of the cavity is fluidically connected with said zone where the propellant is located, a portion of said first hollow body being deformable by a localized impact from one side, a portion of the inner surface of the hollow body being coated with an impact sensitive pyrotechnic material, and
(b.2) means for effecting a mechanical impact on a spot of the outer surface of said hollow body, said spot lying on a point of the outer surface of said hollow body which is opposite to said portion of the inner surface which is coated with an impact sensitive pyrotechnic material, whereby an impact of said spot caused by actuation of said impact effecting means ignites said impact sensitive pyrotechnic material and the products of this ignition ignite the propellant.

39. The device according to claim 37, wherein said means for effecting a mechanical impact comprise a spring mechanism which is arranged for impacting on said spot when a trigger releases a latch.

40. The device according to claim 38, wherein the outlet of the cavity is sealed with a frangible closure that excludes penetration of water vapor into said cavity prior to use of the device.

41. The device according to claim 37, wherein a secondary ignition material is provided and located outside of said cavity and near the outlet of the cavity, so that products of combustion of the impact sensitive pyrotechnic material ignite the secondary ignition material an thereby increase the amount of hot material for igniting the propellant.

42. The device according to claim 38, wherein a secondary ignition material is provided and located inside of said cavity and near the outlet of said cavity, so that products of combustion of the impact sensitive pyrotechnic material ignite the secondary ignition material and thereby increase the amount of hot material for igniting the propellant.

43. The device according to claim 39, wherein one of those surface portions which face each other is coated with an impact sensitive pyrotechnic material and the other is coated with a secondary ignition material.

44. The device according to claim 37, wherein one or both of those surface portions which face each other is coated with a layer of an impact sensitive pyrotechnic material and a layer of a secondary ignition material.

45. The device according to claim 38, said device further comprising
(a) a housing,
(b) a first chamber within said housing, said first chamber containing a medication unit configured and dimensioned to store a volume of liquid medication to be injected, said medication unit having a first region and a second region that are in liquid communication with each other, said first region being deformable and said second region having an ejection outlet, and
(c) a second chamber within said housing, said second chamber containing a propellant,
said first chamber comprising two zones, a first zone containing said medication unit and a second zone which is in communication with said second chamber, so that upon ignition of the propellant in the second chamber gas generated thereby expands into said second zone of said first chamber, exerts pressure on and deforms said deformable first region of said medication unit and thereby causes ejection of said liquid medication through said ejection outlet.

46. The device according to claim 45, wherein said propellant is contained in a propellant chamber having a wall which has a zone of reduced thickness which upon ignition of said propellant bursts and thereby forms an opening of said wall when gas pressure within the propellant chamber exceeds a predetermined value.

47. The device according to claim 37, said device further comprising
(a) a nozzle body, and
(b) a rigid housing,
said housing having a first open end adapted to receive and be connected with the nozzle body and a second closed end,
the interior of said housing defining a chamber which extends between said open end and said closed end of the housing, said chamber being adapted to receive
a first deformable diaphragm which together with a cavity of said nozzle body forms a medication chamber suitable for receiving a predetermined amount of a medication, and
a second deformable diaphragm a portion of which extends around a portion of said first deformable diaphragm,
said second deformable diaphragm and said housing forming together a chamber for receiving a propellant and means for igniting said propellant,
said nozzle body having at its outer end an orifice which is the outlet of a channel for ejecting said medication out of said chamber when a gas pressure generated by ignition of said propellant is applied to said second deformable diaphragm and thereby to said first deformable diaphragm.

48. The device according to claim 47, wherein said nozzle body and said housing are integrally built as a single-piece element and are made of one and the same material.

49. The device according to claim 47, further comprising venting means for venting of the space comprised between the first deformable diaphragm and the second deformable diaphragm.

50. The device according to claim 47, without said second deformable diaphragm.

51. The device according to any of claim 37, said device further comprising
(a) a rigid medication container having a medication zone for receiving said liquid medication,
(b) a nozzle in fluidic communication with said medication zone, said nozzle having an outlet orifice,
(c) a propellant zone where said propellant is located within said device,
(d) a channel that fluidically connects said propellant zone with said medication zone, and
(e) piston means slidably arranged within said channel, so that upon ignition of said propellant gas pressure generated by combustion of the propellant causes displacement of said piston means which then exert pressure on said liquid medication and eject it through the outlet orifice of said nozzle.

52. A device for performing a needleless hypodermic injection of a liquid medication contained in a medication unit within the device, said device including pyrotechnical means for generating within the device a pressure necessary for injecting the medication, said device comprising ignition means for igniting a propellant contained in said device, said ignition means comprising
(a) a pair of hypergolic members formed by a first and a second hypergolic member which are contained within the device, but separated from each other,
said hypergolic members being able to chemically react with each other when brought into contact with each other and which thereby generate heat for igniting said propellant, and
(b) means for bringing said first and a second hypergolic member into contact with each other.

53. A device for performing a needleless hypodermic injection of a liquid medication contained in a medication unit within the device, said device including pyrotechnical means for generating within the device a pressure necessary for injecting the medication, said device comprising ignition means for igniting a propellant contained in said device, said ignition means comprising
(a) a first tube having a closed end and an open end through which the interior of the tube is fluidically connected with a zone within the device where said propellant is located, said tube being deformable by bending it, and
(b) a second tube closed at both ends and arranged within said first tube so that at least a portion of the outer surface of the second tube is spaced from a portion of the inner surface of said first tube, said second tube being breakable by bending the first tube when that bending exceeds a predetermined threshold value,
said second tube containing a first member of an hypergolic pair, and
(c) a second member of an hypergolic pair contained in a space between the inner surface of the first tube and the outer surface of the second tube, whereby when said second tube is broken by bending of the first tube beyond said threshold value the first member and the second member of the hypergolic pair contact each other and their chemical reaction causes an ignition and the products of this ignition ignite the propellant.

54. The device according to claim 53, wherein the outlet of the first tube is sealed with a frangible closure that excludes penetration of water vapor into said first tube prior to use.

55. The device according to claim 53, wherein a secondary ignition material is provided and located outside of said first tube and near the outlet of the first tube, so that products of combustion of the hypergolic pair ignite the secondary ignition material and thereby increase the amount of hot material for igniting the propellant.

56. The device according to claim 53, wherein a secondary ignition material is provided and located inside of said first tube and near the outlet of said first tube, so that products of combustion of the hypergolic pair ignite the secondary ignition material and thereby increase the amount of hot material for igniting the propellant.

57. A device for performing a needleless hypodermic injection of a liquid medication contained in a medication unit within the device, said device including pyrotechnical means for generating within the device a pressure necessary for injecting the medication, said device comprising ignition means for igniting a propellant contained in said device, said ignition means comprising
(a) an elongated container having a closed end and an open end through which the interior of the container is fluidically connected with a zone within the device where said propellant is located, said container being deformable by bending it,
(b) a first tube closed at both ends and arranged within said container, said first tube containing a first member of an hypergolic pair, and
(c) a second tube closed at both ends and arranged within said container, said second tube containing a second member of an hypergolic pair,
said first and second tubes being breakable by bending the container when that bending exceeds a predetermined threshold value, whereby when the first and second tubes are broken by bending of the container beyond said threshold value the first member and the second member of the hypergolic pair contact each other and their chemical reaction causes an ignition and the products of this ignition ignite the propellant.

58. The device according to claim 57, wherein the outlet of said container is sealed with a frangible closure that excludes penetration of water vapor into said first tube prior to use.

59. A device according to claim 57, wherein a secondary ignition material is provided and located near the breaking points of said first and second tubes, so that products of combustion of the hypergolic pair ignite the secondary ignition material and thereby increase the amount of hot material for igniting the propellant.

60. The device according to claim 57, wherein a secondary ignition material is provided and located outside of said container and near the outlet of said container, so that products of combustion of the hypergolic pair ignite the secondary ignition material and thereby increase the amount of hot material for igniting the propellant.

61. The device according to claim 57, wherein a secondary ignition material is provided and located inside of said container and near the outlet of said container, so that products of combustion of the hypergolic pair ignite the secondary ignition material and thereby increase the amount of hot material for igniting the propellant.

62. The device according to claim 52, said device further comprising
(a) a housing,
(b) a first chamber within said housing, said first chamber containing a medication unit configured and dimensioned to store a volume of liquid medication to be injected, said medication unit having a first region and a second region that are in liquid communication with each other, said first region being deformable and said second region having an ejection outlet, and
(c) a second chamber within said housing, said second chamber containing a propellant,
said first chamber comprising two zones, a first zone containing said medication unit and a second zone which is in communication with said second chamber, so that upon ignition of the propellant in the second chamber gas generated thereby expands into said second zone of said first chamber, exerts pressure on and deforms said deformable first region of said medication unit and thereby causes ejection of said liquid medication through said ejection outlet.

63. The device according to claim 62, wherein said propellant is contained in a propellant chamber having a wall which has a zone of reduced thickness which upon ignition of said propellant bursts and thereby forms an opening of said wall when gas pressure within the propellant chamber exceeds a predetermined value.

64. The device according to claim 52, said device further comprising
(a) a nozzle body, and
(b) a rigid housing,
said housing having a first open end adapted to receive and be connected with the nozzle body and a second closed end,
the interior of said housing defining a chamber which extends between said open end and said closed end of the housing, said chamber being adapted to receive
a first deformable diaphragm which together with a cavity of said nozzle body forms a medication chamber suitable for receiving a predetermined amount of a medication, and
a second deformable diaphragm a portion of which extends around a portion of said first deformable diaphragm,
said second deformable diaphragm and said housing forming together a chamber for receiving a propellant and means for igniting said propellant,
said nozzle body having at its outer end an orifice which is the outlet of a channel for ejecting said medication out of said chamber when a gas pressure generated by ignition of said propellant is applied to said second deformable diaphragm and thereby to said first deformable diaphragm.

65. The device according to claim 64, wherein said nozzle body and said housing are integrally built as a single-piece element and are made of one and the same material.

66. The device according to claim 64, further comprising venting means for venting of the space comprised between the first deformable diaphragm and the second deformable diaphragm.

67. The device according to claim 64, without said second deformable diaphragm.

68. The device according to claim 52, said device further comprising
(a) a rigid medication container having a medication zone for receiving said liquid medication,
(b) a nozzle in fluidic communication with said medication zone, said nozzle having an outlet orifice,
(c) a propellant zone where said propellant is located within said device,
(d) a channel that fluidically connects said propellant zone with said medication zone, and
(e) piston means slidably arranged within said channel, so that upon ignition of said propellant gas pressure generated by combustion of the propellant causes displacement of said piston means which then exert pressure on said liquid medication and eject it through the outlet orifice of said nozzle.

69. A device for performing a needleless hypodermic injection of a liquid medication contained in a medication unit within the device, said device including pyrotechnical means for generating within the device a pressure necessary for injecting the medication, said device comprising ignition means for igniting a propellant contained in said device, said ignition means comprising
(a) a rotatable body,
(b) a stationary body,
(c) means for pressing at least a portion of said rotatable body against said stationary body, a portion of the outer surface of said rotatable body being thereby in contact with said stationary body and
at least a part of said portion of the outer surface of said rotatable body or at least a part of the outer surface of said stationary body being coated with a combustible mixture that bursts into flame when activated by friction,
(d) locking means that when locked prevent rotation of said rotatable body, and
(e) means for rotating said rotatable body when said locking means are unlocked, said rotation causing a friction of said combustible mixture against an outer surface of said stationary body or an outer surface of said stationary body and thereby ignition of said mixture, the products of this ignition igniting the propellant.

70. A device for performing a needleless hypodermic injection of a liquid medication contained in a medication unit within the device, said device including pyrotechnical means for generating within the device a pressure necessary for injecting the medication, said device comprising ignition means for igniting a propellant contained in said device, said ignition means comprising
(a) a rotatable body,
(b) a stationary body,
(c) means for pressing at least a portion of said rotatable body against said stationary body, a portion of the outer surface of said rotatable body being thereby in contact with said stationary body and
at least a part of said portion of the outer surface of said rotatable body being coated with a first member of an hypergolic pair, and at least a part of the outer surface of said stationary body being coated with a second member of an hypergolic pair,
(d) locking means that when locked prevent rotation of said rotatable body,
(e) means for rotating said rotatable body when said locking means are unlocked, said rotation bringing said first and second hypergolic member into contact with each other, thereby causing their chemical reaction and a resulting ignition, the products of this ignition igniting the propellant.

71. The device according to claim 69, wherein said rotatable body and said stationary body are in a zone closed by a frangible closure that protects coatings on those bodies against effects of humidity.

72. The device according to claim 69, wherein a secondary ignition material is provided and located near to the zone where the rotatable body and the stationary body contact each other.

73. The device according to claim 69, said device further comprising
(a) a housing,
(b) a first chamber within said housing, said first chamber containing a medication unit configured and dimensioned to store a volume of liquid medication to be injected, said medication unit having a first region and a second region that are in liquid communication with each other, said first region being deformable and said second region having an ejection outlet, and
(c) a second chamber within said housing, said second chamber containing a propellant,
said first chamber comprising two zones, a first zone containing said medication unit and a second zone which is in communication with said second chamber, so that upon ignition of the propellant in the second chamber gas generated thereby expands into said second zone of said first chamber, exerts pressure on and deforms said deformable first region of said medication unit and thereby causes ejection of said liquid medication through said ejection outlet.

74. The device according to claim 73, wherein said propellant is contained in a propellant chamber having a wall which has a zone of reduced thickness which upon ignition of said propellant bursts and thereby forms an opening of said wall when gas pressure within the propellant chamber exceeds a predetermined value.

75. The device according to claim 69, said device further comprising
(a) a nozzle body, and
(b) a rigid housing,
said housing having a first open end adapted to receive and be connected with the nozzle body and a second closed end,
the interior of said housing defining a chamber which extends between said open end and said closed end of the housing, said chamber being adapted to receive
a first deformable diaphragm which together with a cavity of said nozzle body forms a medication chamber suitable for receiving a predetermined amount of a medication, and
a second deformable diaphragm a portion of which extends around a portion of said first deformable diaphragm,
said second deformable diaphragm and said housing forming together a chamber for receiving a propellant and means for igniting said propellant,
said nozzle body having at its outer end an orifice which is the outlet of a channel for ejecting said medication out of said chamber when a gas pressure generated by ignition of said propellant is applied to said second deformable diaphragm and thereby to said first deformable diaphragm.

76. The device according to claim 75, wherein said nozzle body and said housing are integrally built as a single-piece element and are made of one and the same material.

77. The device according to claim 75, further comprising venting means for venting of the space comprised between the first deformable diaphragm and the second deformable diaphragm.

78. The device according to claim 75, without said second deformable diaphragm.

79. The device according to claim 69, said device further comprising
(a) a rigid medication container having a medication zone for receiving said liquid medication,
(b) a nozzle in fluidic communication with said medication zone, said nozzle having an outlet orifice,
(c) a propellant zone where said propellant is located within said device,
(d) a channel that fluidically connects said propellant zone with said medication zone, and
(e) piston means slidably arranged within said channel, so that upon ignition of said propellant gas pressure generated by combustion of the propellant causes displacement of said piston means which then exert pressure on said liquid medication and eject it through the outlet orifice of said nozzle.

80. A device for performing a needleless hypodermic injection of a liquid medication contained in a medication unit within the device, said device including pyrotechnical means for generating within the device a pressure necessary for injecting the medication, said device comprising ignition means for igniting a propellant contained in said device, said device further comprising
(a) a housing which is so configured and dimensioned that it is adapted to withstand or uptake alone, i.e. by itself, said predetermined internal pressure value,
(b) a first chamber within said housing, said first chamber containing a medication unit configured and dimensioned to store a volume of liquid medication to be injected, said medication unit having a first region and a second region that are in liquid communication with each other, said first region being deformable and said second region having an ejection outlet,
(c) a second chamber within said housing, said second chamber containing a propellant,
said first chamber comprising two zones, a first zone containing said medication unit and a second zone which is in communication with said second chamber, so that upon ignition of the propellant in the second chamber gas generated thereby expands into said second zone of said first chamber, exerts pressure on and deforms said deformable first region of said medication unit and thereby causes ejection of said liquid medication through said ejection outlet, and
(d) ignition means comprising a piezoelectric spark generator.

81. The device according to claim 80, wherein said propellant is contained in a propellant chamber having a wall which has a zone of reduced thickness which upon ignition of said propellant bursts and thereby forms an opening of said wall when gas pressure within the propellant chamber exceeds a predetermined value.

82. A device for performing a needleless hypodermic injection of a liquid medication contained in a medication unit within the device, said device including pyrotechnical means for generating within the device a pressure necessary for injecting the medication, said device comprising ignition means for igniting a propellant contained in said device, said device further comprising
(a) a nozzle body, and
(b) a rigid housing,
said housing having a first open end adapted to receive and be connected with the nozzle body and a second closed end,
the interior of said housing defining a chamber which extends between said open end and said closed end of the housing, said chamber being adapted to receive
a first deformable diaphragm which together with a cavity of said nozzle body forms a medication chamber suitable for receiving a predetermined amount of a medication, and
a second deformable diaphragm a portion of which extends around a portion of said first deformable diaphragm,
said second deformable diaphragm and said housing forming together a chamber for receiving a propellant and means for igniting said propellant,
said nozzle body having at its outer end an orifice which is the outlet of a channel for ejecting said medication out of said chamber when a gas pressure generated by ignition of said propellant is applied to said second deformable diaphragm and thereby to said first deformable diaphragm
said means for igniting said propellant comprising a piezoelectric spark generator.

83. The device according to claim 82, wherein said nozzle body and said housing are integrally built as a single-piece element and are made of one and the same material.

84. The device according to claim 82, further comprising venting means for venting of the space comprised between the first deformable diaphragm and the second deformable diaphragm.

85. The device according to claim 82, without said second deformable diaphragm.

86. A device for performing a needleless hypodermic injection of a liquid medication contained in a medication unit within the device, said device including pyrotechnical means for generating within the device a pressure necessary for injecting the medication, said device comprising ignition means for igniting a propellant contained in said device, said device further comprising
(a) a rigid medication container having a medication zone for receiving said liquid medication,
(b) a nozzle in fluidic communication with said medication zone, said nozzle having an outlet orifice,
(c) a propellant zone where said propellant is located within said device,
(d) a channel that fluidically connects said propellant zone with said medication zone,
(e) piston means slidably arranged within said channel, so that upon ignition of said propellant gas pressure generated by combustion of the propellant causes displacement of said piston means which then exert pressure on said liquid medication and eject it through the outlet orifice of said nozzle and
(f) ignition means for igniting said propellant, said ignition means comprising a piezoelectric spark generator.

87. A device for performing a needleless hypodermic injection of a liquid medication according to any of the preceding claims, wherein said propellant is a one-piece propellant pellet.

88. The device according to claim 87, wherein an ignition layer is in contact with or is an integral part of said one-piece propellant pellet.

89. The device according to claim 87, wherein said propellant is an array of one-piece propellant pellets having each a predetermined shape, a predetermined chemical composition and a predetermined relative position within the array.

90. The device according to claim 89, wherein an ignition layer is in contact with or is an integral part of said array of one-piece propellant pellets.
